(19) United States
(12) Patent Application Publication
(10) Pub. No.: US 2009/0252687 A1
(43) Pub. Date: Oct. 8, 2009

(54) ASYMMETRIC FLUORO-SUBSTITUTED POLYMETHINE DYES

(76) Inventor: Michael Edward Cooper, Cardiff (GB)

Correspondence Address: GE HEALTHCARE, INC., IP DEPARTMENT 101 CARNEGIE CENTER, PRINCETON, NJ, 08540-6231, US

(21) Appl. No.: 12/375,239

(22) PCT Filed: Jul. 31, 2007
(86) PCT No.: PCT/GB07/02907

§ 371 Date: Jan. 27, 2009

(30) Foreign Application Priority Data
Jul. 31, 2006 (GB) 0615211.0

(51) Int. Cl. A61B 10/00; A61K 49/00; G01N 21/76; C09B 23/08

(52) U.S. Cl. 424/9.6; 436/172; 525/54.1

(57) ABSTRACT

The present invention relates to improved conjugates of biological molecules with an improved class of water-soluble, green to near infra-red (NIR) cyanine labelling dyes. The dyes are asymmetric fluoro-substituted polymethines, and exhibit a high degree of photostability and reduced dye-dye quenching, as well as a high fluorescence quantum yield. The conjugates are useful for in vivo optical imaging, as well as fluorescence detection methods. Also disclosed are pharmaceutical compositions containing the conjugates, kits for the preparation of such compositions, and methods of in vivo imaging using the conjugates.

### FIELD OF THE INVENTION

The present invention relates to improved conjugates of biological molecules with an improved class of water-soluble, red to near infra-red (NIR) cyanine labelling dyes. The dyes are asymmetric fluoro-substituted polymethines, and exhibit a high degree of photostability and reduced dye-dye quenching, as well as a high fluorescence quantum yield. The conjugates are useful for in vivo optical imaging, as well as fluorescence detection methods. Also disclosed are pharmaceutical compositions containing the conjugates, kits for the preparation of such compositions, and methods of in vivo imaging using the conjugates.

### BACKGROUND TO THE INVENTION

Fluorescent dyes based on polymethine chromophores are characterised by strong absorption maxima which can extend over a wide wavelength range. U.S. Pat. No. 6,048,982 (Waggoner) discloses luminescent cyanine dyes having the structure (1):

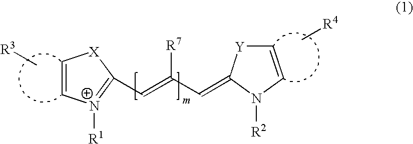

wherein X and Y are independently selected from the group consisting of O, S and CH₃—C—CH₃; m is an integer from 1-4 and at least one of the groups R¹, R², R³, R⁴ and R⁷ is a reactive group, reactive with amino, sulphydryl or hydroxy nucleophiles. In cyanine dyes of the above formula, the number of methine groups linking the heterocyclic ring systems defines the absorption and emission maxima of the dyes. Thus, the absorption maxima increase from 3 to 5 to 7 methine groups (Cy^{TM}3 to Cy5 to Cy7 respectively) by an increment of approximately 100 nm each. The corresponding emission peaks of Cy3, Cy5 and Cy7 are also separated by approximately 100 nm. For dyes to be useful in the NIR range it is necessary to assemble at least 5 sp² carbon atoms linking the heterocyclic bases (pentamethine dyes), thereby providing dyes with a wavelength Em (max) of at least 650 nm. Lengthening the polymethine chain in such dyes is accompanied by an increase in the opportunity for chemical (nucleophilic or electrophilic) attack on the chain, leading to a loss of conjugation, a decrease in dye photostability, and low fluorescence quantum yields. Thus, fluorescence dyes which contain increased “heteroaromaticity” are to be expected to offer advantages over conventional polymethine dyes having the same number of methine groups.

Laser-compatible NIR marker dyes based on benzopyrilium polymethines have been described in U.S. Pat. No. 6,750,346 (Czerney, P. et al) which discloses inter alia a compound of structure (2):

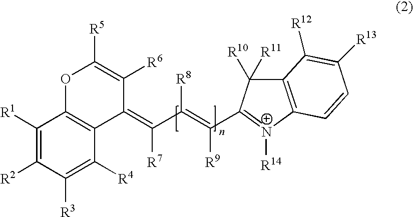

in which groups R¹ to R¹⁴ are equal or different and represent in each case H, Cl, Br, an aliphatic or mononuclear aromatic group, each having at most 12 carbon atoms which may contain as a substituted group in addition to carbon and hydrogen, up to 4 oxygen atoms and 0, 1 or 2 nitrogen atoms or a sulfur atom, or a sulfur and a nitrogen atom or represents an amino function, having a nitrogen atom to which there is bound, hydrogen or at least one substituent having up to 8 carbon atoms, the substituent selected from the group consisting of carbon, hydrogen and up to two sulfonic acid groups, and n is zero, 1 or 2. At least one of R¹ to R¹⁴ may contain a reactive group.

Recently, Waggoner et al [Org.Lett., 6(6), 909-912 (2004)] described a polyfluorothiadicarbocyanine dye (3) having good photostability in aqueous solvents. The dye exhibited reduced aggregation, enhanced quantum yield and greater resistance to photobleaching when compared with a non-fluorinated analogue:

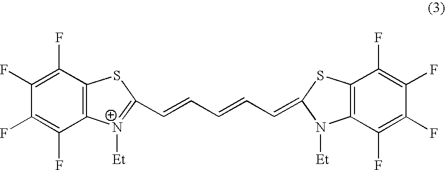

Modification of the indolium ring of a carbocyanine dye at least one of the 3-positions, so as to introduce a reactive group or a conjugated substance has been described in WO 02/26891 (Molecular Probes Inc.). The modified dyes according to WO 02/26891 have also been reported to overcome the tendency of cyanine dyes to self-associate and dye conjugates labelled with the modified dyes are reported to be more fluorescent than conjugates labelled with structurally similar carbocyanine dyes.

Vompe et al [Proc.USSR Acad.Sci., 272(3), 615-618 (1983)] disclose unsymmetrical heptamethine dyes having an indoline heterocyclic ring at one end of the methine chain, and the other ring chosen from benzoxazole, thiazoline, pyrroline, 4-quinoline or benzimidazole. The ring may optionally be substituted with a CF₃ group. The paper is silent on conjugates of the dyes with biomolecules.

### THE PRESENT INVENTION

None of the above documents discloses conjugates of a biological targeting moiety with asymmetric polymethine dyes containing one or preferably multiple fluoro substituents attached to an indocyanine chromophore as are described herein.

Furthermore, the dyes of the present invention possess one or more sulfonic acid groups attached to the 1- or 3-position of the indolium ring system. The present dyes exhibit increased photostability, and reduced dye-dye interactions. The increased photostability and reduced dye-dye interaction resulting in enhanced brightness are particularly useful for in-vivo applications, as for example in endoscopic imaging, where the light intensity and the related photobleaching are high and the informative fluorescent signal being limited should be separated from the unfavourable tissue autofluorescence and background signal. It is anticipated that the improved chemical stability of the present dyes will confer improved resistance to various biochemical processes that occur within living organism (such as enzymatic degradation), that will potentially reduce the toxicity and/or imaging inefficacy due to the metabolism of the dye-[biological targeting moiety] conjugate. The dyes are also useful for in vitro assays involving fluorescence detection where continual excitation is a requirement, for example in kinetic studies, or in microarray analyses where microarray slides may need to be reanalysed over a period of days.

### DETAILED DESCRIPTION OF THE INVENTION

In a first aspect the present invention provides a dye conjugate of Formula I:

[BTM]-(L)ⱼ-Cy^{D} (I)

where:
BTM is a biological targeting moiety;
Cy^{D} is a cyanine dye of Formula II:

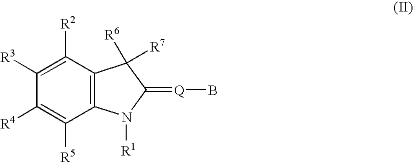

wherein:
Q is a group containing 1, 2 or 3 carbon-carbon double bonds which forms a conjugated system with B;
R¹, R⁶ and R⁷ are selected independently from C₁₋₄ alkyl or —(CH₂)ₖ—SO₃M¹;
R², R³, R⁴ and R⁵ are selected independently from H, F, —SO₃M¹ and —(CF₂)ₘ—F, where m is an integer of value 1 to 4;
M¹ is H or BC, where Bc is a biocompatible cation;
j is 0 or 1;
k is an integer of value 1 to 10;
L is a synthetic linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
B is an aromatic chromophore selected from benzo[b]pyrilium, quinolinium and acridinium chromophores;
   with the proviso that at least one of R², R³, R⁴ and R⁵ is F or —(CF₂)ₘ—F.

“Alkyl” is a straight or branched chain alkyl group containing from 1-4 carbon atoms, for example methyl, ethyl, n-propyl, iso-propyl and n-butyl and t-butyl.

“Aryl” is an aromatic substituent containing one or two fused aromatic rings containing 6 to 10 carbon atoms, for example phenyl or naphthyl, the aryl being optionally and independently substituted by one or more substituents, for example halogen, C₁₋₄ alkyl or C₁₋₄ alkoxyl.

“Alkoxyl” is a C₁-4 alkoxy substituent for example methoxy, ethoxy, propoxy and n-butoxy.

“Heteroaryl” is a mono- or bicyclic 5- to 10-membered aromatic ring system containing at least one heteroatom which may be selected from N, O, and S and is optionally and independently substituted by one or more substituents, for example halogen, straight or branched C₁-4 alkyl or C₁-4 alkoxyl.

“Aralkyl” is a C₁-4 alkyl group substituted by an aryl or heteroaryl group as hereinbefore defined.

“Halogen” and halo groups are selected from fluorine, chlorine, bromine and iodine.

By the term “biocompatible cation” (BC) is meant a positively charged counterion which forms a salt with an ionised, negatively charged group (in this case a sulfonate group), where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “biological targeting moiety” (BTM) is meant a compound which, after administration, is taken up selectively or localises at a particular site of the mammalian body in vivo. Such sites may for example be implicated in a particular disease state, or be indicative of how an organ or metabolic process is functioning. The BTM preferably comprises: 3-100 mer peptides or peptide analogues which may be linear peptides or cyclic peptides or combinations thereof, or enzyme substrates, enzyme antagonists or enzyme inhibitors; synthetic receptor-binding compounds; oligonucleotides, or oligo-DNA or oligo-RNA fragments.

By the term “peptide” is meant a compound comprising two or more amino acids, as defined below, linked by a peptide bond (i.e. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one of more amino acid analogues, as described below.

By the term “amino acid” is meant an L- or D-amino acid, amino acid analogue (e.g. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable enzyme substrates, antagonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

In another embodiment, particularly for in vitro applications, the BTM may be an affinity tag which is capable of binding specifically and non-covalently with its complementary specific binding partner, thereby forming a specific binding pair. Examples of specific binding pairs include, but are not restricted to: biotin/avidin, biotin/streptavidin, polyhistidine tag-metal ion complexes with nitrilotriacetic acid (e.g. Ni²⁺: NTA). The complementary specific binding partner may be one component of a labelling complex for detection of a target component. It is to be understood that in the context of the present invention, any two atoms or molecules that possess a specific binding affinity one for the other, may be employed. Preferred examples of affinity tags are selected from biotin, iminobiotin and desthiobiotin.

The cyanine dye (Cy^{D}) of Formula II is a fluorescent dye or chromophore which is capable of detection either directly or indirectly in an optical imaging procedure using light of green to near-infrared wavelength (500-1200 nm, preferably 600-1000 nm). Preferably, the Cy^{D} has fluorescent properties.

Preferably Q is the group:

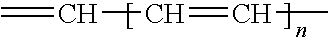

wherein n=1, 2 or 3. Preferred NIR dyes are those in which n is selected to be 2 or 3, most preferably 2.

j is preferably 1, i.e. a linking group (L) is present. It is envisaged that one of the roles of the linker group -(A)ₘ- of Formula I is to distance the Cy^{D} from the active site of the BTM. This is particularly important because the CYD is relatively bulky, so undesirable steric interactions could otherwise occur. This can be achieved by a combination of flexibility (e.g. simple alkyl chains), so that the CYD has the freedom to position itself away from the active site and/or rigidity such as a cycloalkyl or aryl spacer which orientate the Cy^{D} away from the active site. The nature of the linker group can also be used to modify the biodistribution of an in vivo imaging agent. Thus, e.g. the introduction of ether groups in the linker will help to minimise plasma protein binding. When -(A)ₘ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, e.g. via the kidneys as opposed to via the liver.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, e.g. a glucosamine derivative of an amino acid can be conjugated to other amino acids via peptide bonds. The glucosamine derivative of asparagine (commercially available from NovaBiochem) is one example of this:

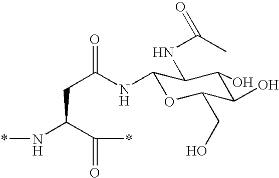

Formula I denotes that the -(L)ⱼ[Cy^{D}] moiety can be attached at any suitable position of the BTM. Suitable such positions for the -(L)ⱼ[Cy^{D}] moiety are chosen to be at positions away from that part of the BTM which is responsible for binding to the active site in vivo.

Suitably B is of Formula IIa:

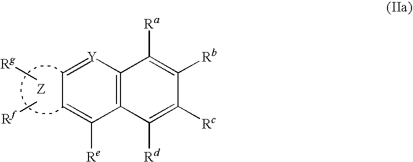

wherein:
Y is selected from O⁺ and N⁺—R⁸, where R⁸ is selected from H, C₁₋₄ alkyl and —(CH₂)ₖ—SO₃M¹;
R^{a}, R^{b}, R^{c}, R^{d}, R^{e}, R^{f} and R^{g} are selected independently from Q, H, C₁₋₄ alkyl, C₆₋₁₀ aryl, heteroaryl, aralkyl, Hal, sulphydryl, amino, C₁₋₄ alkyl-substituted amino, quaternary ammonium, —SO₃M¹, —OR⁹ and —COOR⁹, where R⁹ is selected from H and C₁₋₄ alkyl;
Z represents an optional fused phenyl ring, such that R^{f} and R^{g} are attached to the Z ring when Z is present, or the Y ring when Z is absent;
   with the proviso that one of R^{a}, R^{b}, R^{c}, R^{d}, R^{e}, R^{f} and R^{g} is Q.

In Formula IIa, R⁸ is preferably —(CH₂)ₖ—SO₃M¹.

One preferred embodiment of Formula IIa is where B is a benzo[b]pyrilium chromophore, such that Y is O⁺, Z is absent, and R^{e} is the preferred Q group described above, said dye having the Formula (III):

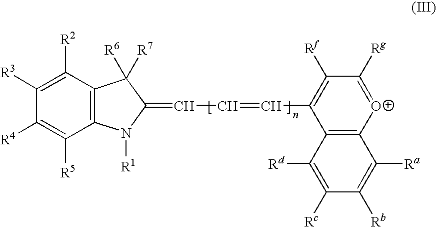

In Formula III, R^{b} is preferably an amino group of formula —NR¹⁰R¹¹, where R¹⁰ and R¹¹ are independently H or C₁₋₄ alkyl, or wherein R¹⁰ in combination with R^{a} or R¹¹ in combination with R^{c}, or both, form additional saturated or unsaturated 6-membered rings. Preferably, R^{g} of Formula III is C₁-C₄ alkyl, for example methyl, ethyl, n-propyl, iso-propyl, n-butyl and t-butyl.

A second preferred embodiment of Formula Ia is where B comprises a quinolinium chromophore such that Y is N⁺—R⁸, Z is absent, together with the preferred Q group, said dye having a structure chosen from Formula (IVa) or (IVb) or (IVc):

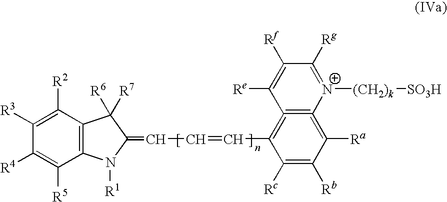

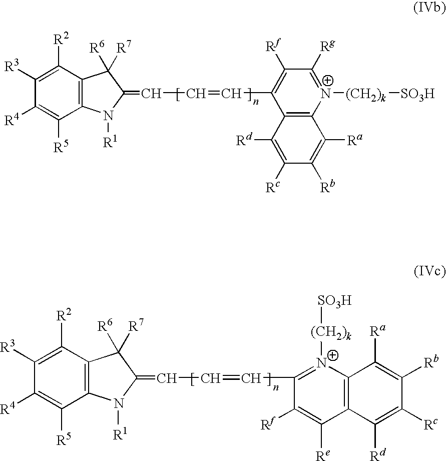

A third preferred embodiment of Formula IIa is where B is an acridinium chromophore such that Y is N⁺—R⁸, Z is present, together with the preferred Q group as defined above, said dye having the Formula (V):

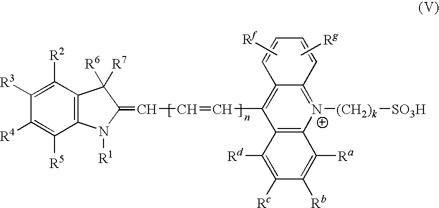

### Preferred Features.

The compounds of the present invention comprise at least one, preferably two or more fluorine atoms substituted directly or indirectly onto the dye chromophore. In one embodiment, compounds of Formulae (II), (III), (IVa), (IVb), (IVc) and (V) may be substituted in the indolium ring system by a fluorine atom. Hence, for the R², R³, R⁴ and R⁵ groups, preferably at least one, more preferably at least two, and most preferably at least three are chosen to be F. Any remaining groups R², R³, R⁴ and R⁵ are preferably H. In a particularly preferred embodiment, each of R², R³, R⁴ and R⁵ is F. Fluoro substitution of the dyes of the present invention has been found to improve dye photostability.

In another embodiment, the compounds of Formulae (II), (III), (IVa), (IVb), (IVc) and (V) may include a C₁₋₄ perfluoroalkyl substituent of formula —(CF₂)ₘF, where m is an integer of value 1 to 4, in the indolium ring system at one, preferably not more than two of the R², R³, R⁴ and R⁵ positions. The remaining groups R², R³, R⁴ and R⁵ are preferably selected from H or F. Preferably, the perfluoroalkyl substituent is trifluoromethyl, i.e. m is 1.

The Cy^{D} dyes of the present invention may be substituted directly or indirectly with from two to four or more sulfonic acid groups, preferably between two and three sulfonic acid groups. These sulfonic acid groups are chosen from the —SO₃M¹ and —(CH₂)ₖSO₃M¹ substituents of Formula II. The use of dyes substituted by fluorine and having three or more sulfonic acid groups for labelling BTM results in a labelled product in which there is reduced dye-dye aggregation and improved photostability, compared with dyes having no such substitutions. The fluorescence emission intensity of a BTM so labelled with the preferred dyes of the present invention increases with the number of covalently attached dyes. Furthermore, substitution of the indolinium 3-position with sulfonic acid groups in addition to increasing the overall charge on the dye molecule, also adds steric bulk, thereby contributing to a reduction in dye-dye aggregation. In Formulae (II), (III), (IVa), (IVb), (IVc) and (V), preferred groups —(CH₂)ₖ—SO₃M¹ are those in which k is 3 or 4, that is —(CH₂)₃SO₃M¹ and —(CH₂)₄SO₃M¹.

In Formula I, the [BTM]-(L)ⱼ- moiety is covalently attached at any suitable position of the Cy^{D}, including B, Q or the R¹ to R⁷ groups. The [BTM]-(L)ₙ- moiety either takes the place of an existing substituent (e.g. the R¹ to R⁷ groups), or is covalently attached to the existing substituent. Preferably, the [BTM]-(L)ⱼ- moiety is attached at one or more of positions R¹, R⁶, R⁷, R⁸, R^{a}, R^{b}, R^{c}, R^{d}, R^{e}, R^{f} and R^{g} of the Cy^{D} of Formulae (II), (III), (IVa), (IVb), (IVc) and (V). The [BTM]-(L)ⱼ- moiety is more preferably attached at one or more of the R¹, R⁶ or R⁷ positions of the Cy^{D}. In one most preferred embodiment, R¹ is -(L)ⱼ[BTM]; and one of R⁶ and R⁷ is —(CH₂)ₖ—SO₃M¹ and the other is C₁₋₄ alkyl. In a second most preferred embodiment, R¹ is —(CH₂)ₖ—SO₃M¹; and one of R⁶ and R⁷ is -(L)ⱼ[BTM] and the other is C₁₋₄ alkyl. The BTM may be of synthetic or natural origin, but is preferably synthetic. The term “synthetic” has its conventional meaning, i.e. man-made as opposed to being isolated from natural sources e.g. from the mammalian body. Such compounds have the advantage that their manufacture and impurity profile can be fully controlled. Monoclonal antibodies and fragments thereof of natural origin are therefore outside the scope of the term ‘synthetic’ as used herein.

The BTM is preferably chosen from: a 3-100 mer peptide, enzyme substrate, enzyme antagonist or enzyme inhibitor. BTM is most preferably a 3-100 mer peptide or peptide analogue. When the BTM is a peptide, it is preferably a 4-30 mer peptide, and most preferably a 5 to 28-mer peptide.

When the BTM is a peptide, preferred such peptides include:
somatostatin, octreotide and analogues,
peptides which bind to the ST receptor, where ST refers to the heat-stable toxin produced by E. coli and other micro-organisms;
laminin fragments e.g. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may e.g. target angiogenesis [R. Pasqualini et al., Nat Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J. Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044)
[Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu;

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase, metabolism of the BTM peptide at either the amino terminus or carboxy terminus. Such groups are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described for the linker group (L), below. Preferred such PEG groups are the biomodifiers of Formulae Bio1 or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

When either or both peptide termini are protected with an M^{IG} group, the -(L)ⱼ[Cy^{D}] moiety may optionally be attached to the M^{IG} group. Preferably, at least one peptide terminus has no M^{IG} group, so that attachment of the -(L)ⱼ[Cy^{D}] moiety at that position gives compounds of Formulae Va or Vb respectively:

[Cy^{D}]-(L)ⱼ-[BTM]-Z² (Va);

Z¹-[BTM]-(L)ⱼ-[Cy^{D}] (Vb);

where:
Z¹ is attached to the N-terminus of the BTM peptide, and is H or M^{IG};
Z² is attached to the C-terminus of the BTM peptide and is OH, OB^{c}, or M^{IG}, where B^{c} is a biocompatible cation (as defined above).

In Formula Va and Vb, Z¹ and Z² are preferably both independently M^{IG}. Preferred such M^{IG} groups for Z¹ and Z² are as described above for the peptide termini. Whilst inhibition of metabolism of the BTM peptide at either peptide terminus may also be achieved by attachment of the -(L)ⱼ[Cy^{D}] moiety in this way, -(L)ⱼ[Cy^{D}] itself is outside the definition of M^{IG} of the present invention.

The BTM peptide may optionally comprise at least one additional amino acid residue which possesses a side chain suitable for facile conjugation of the Cy^{D}, and forms part of the (A)ₘ residues of the linker group (L). Suitable such amino acid residues include Asp or Glu residues for conjugation with amine-functionalised Cy^{D} dyes, or a Lys residue for conjugation with a carboxy- or active ester-functionalised Cy^{D} dye. The additional amino acid residue(s) for conjugation of Cy^{D} are suitably located away from the binding region of the BTM peptide, and are preferably located at either the C- or N-terminus. Preferably, the amino acid residue for conjugation is a Lys residue.

When a synthetic linker group (L) is present, it preferably comprises terminal functional groups which facilitate conjugation to [BTM] and Cy^{D}. Suitable such groups (Q^{a}) are described below. When L comprises a peptide chain of 1 to 10 amino acid residues, the amino acid residues are preferably chosen from glycine, lysine, arginine, aspartic acid, glutamic acid or serine. For in vivo applications, in one embodiment when L comprises a PEG moiety, it preferably comprises units derived from oligomerisation of the monodisperse PEG-like structures of Formulae Bio1 or Bio2:

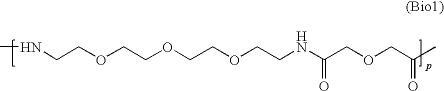

17-amino-5-oxo-6-aza-3,9,12,15-tetraoxaheptadecanoic acid of Formula Bio1

wherein p is an integer from 1 to 10. Alternatively, a PEG-like structure based on a propionic acid derivative of Formula Bio2 can be used:

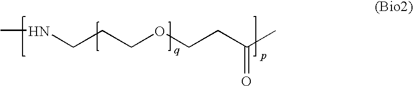

where p is as defined for Formula Bio1 and q is an integer from 3 to 15.

In Formula Bio2, p is preferably 1 or 2, and q is preferably 5 to 12.

In a second embodiment for in vivo applications, when the linker group does not comprise PEG or a peptide chain, preferred L groups have a backbone chain of linked atoms which make up the -(A)ₘ- moiety of 2 to 10 atoms, most preferably 2 to 5 atoms, with 2 or 3 atoms being especially preferred. A minimum linker group backbone chain of 2 atoms confers the advantage that the Cy^{D} is well-separated so that any undesirable interaction is minimised.

For in vitro applications, the linking group L is preferably selected from:

—(CHR′)ₚ-M-(CHR′)ᵣ—

where M is selected from: —CHR′—, —NR′—, —O—, —S—, —Ar—, —C(O)—NR′— and —C(O)—O—; R′ is H or C₁₋₄ alkyl, Ar is phenyl, optionally substituted with sulphonate, p and r are integers of value 1-5. Particularly preferred linking groups are those wherein M is selected from —CH₂— and —CONH—.

BTM peptides which are not commercially available can be synthesised by solid phase peptide synthesis as described in P. Lloyd-Williams, F. Albericio and E. Girald; Chemical Approaches to the Synthesis of Peptides and Proteins, CRC Press, 1997.

In a second aspect, the present invention provides a pharmaceutical composition which comprises the conjugate of the first aspect together with a biocompatible carrier, in a form suitable for mammalian administration.

The “biocompatible carrier” is a fluid, especially a liquid, in which the conjugate can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection or isotonic saline.

The conjugate and biocompatible carrier are each supplied in suitable vials or vessels which comprise a sealed container which permits maintenance of sterile integrity and/or radioactive safety, plus optionally an inert headspace gas (e.g. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (e.g. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the second aspect may be prepared under aseptic manufacture (i.e. clean room) conditions to give the desired sterile, non-pyrogenic product. It is preferred that the key components, especially the associated reagents plus those parts of the apparatus which come into contact with the conjugate (e.g. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition of the second aspect is preferably prepared from a kit, as described for the third aspect below.

In a third aspect, the present invention provides a kit for the preparation of the pharmaceutical composition of the second aspect, said kit comprising the conjugate of the first aspect in sterile, solid form such that, upon reconstitution with a sterile supply of a biocompatible carrier as described in the second aspect, dissolution occurs to give the desired pharmaceutical composition.

In that instance, the conjugate, plus other optional excipients as described above, may be provided as a lyophilised powder in a suitable vial or container. The lyophilisate is then designed to be reconstituted with the desired biocompatible carrier to the pharmaceutical composition in a sterile, apyrogenic form which is ready for mammalian administration.

A preferred sterile, solid form of the conjugate is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a fourth aspect, the present invention provides a functionalised dye useful in the preparation of the conjugate of the first aspect. Said functionalised dye comprises the Cy^{D} of Formula II wherein said Cy^{D} further comprises a group Q^{a}, wherein Q^{a} is a reactive functional group suitable for conjugation to the BTM. The Q^{a} group is designed to react with a complementary functional group of the BTM, thus forming a covalent linkage between the Cy^{D} and the BTM. The complementary functional group of the BTM may be an intrinsic part of the BTM, or may be introduced by derivatisation of the BTM with a bifunctional compound as is known in the art. Preferably, the BTM is used without derivatisation, so that the Q^{a} group is preferably a reactive group.

Table 1 shows examples of reactive groups and their complementary counterparts:

TABLE 1
Reactive Substituents and Complementary
Groups Reactive Therewith.

| Reactive Groups | Complementary Groups |
|---|---|
| Activated ester, | primary amino, secondary amino |
| eg. succinimidyl ester, | |
| sulpho-succinimidyl ester, | |
| or pentafluorophenyl. | |
| acid anhydride, acid halide. | primary amino, secondary amino, hydroxyl |
| isothiocyanate | amino groups |
| vinylsulphone | amino groups |
| dichlorotriazine | amino groups |
| haloacetamide, maleimide | thiol, imidazole, hydroxyl, amines, |
| | thiophosphate |
| carbodiimide | carboxylic acids |
| hydrazine, hydrazide | carbonyl including aldehyde and ketone |
| phosphoramidite | hydroxyl groups |

By the term “activated ester” or “active ester” is meant an ester derivative of the carboxylic acid which is designed to be a better leaving group, and hence permit more facile reaction with nucleophile, such as amines. Examples of suitable active esters are: N-hydroxysuccinimide (NHS), pentafluorophenol, pentafluorothiophenol, para-nitrophenol and hydroxybenzotriazole. Preferred active esters are N-hydroxysuccinimide or pentafluorophenol esters.

Examples of functional groups present in BTM such as proteins, peptides, nucleic acids carbohydrates and the like, include: hydroxy, amino, sulphydryl, carbonyl (including aldehyde and ketone) and thiophosphate. Suitable Q^{a} groups may be selected from: carboxyl; activated esters; isothiocyanate; maleimide; haloacetamide; hydrazide; vinylsulphone, dichlorotriazine and phosphoramidite. Preferably, Q^{a} is: an activated ester of a carboxylic acid, an isothiocyanate, a maleimide or a haloacetamide.

When the complementary group is an amine or hydroxyl, Q^{a} is preferably an activated ester, with especially preferred such esters including:

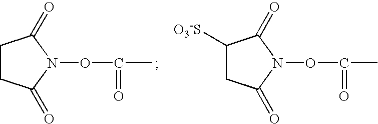

A preferred such substituent on the Cy^{D} is the activated ester of an alkyl carboxylic acid, preferably a 5-carboxypentyl group. Preferred such esters include:

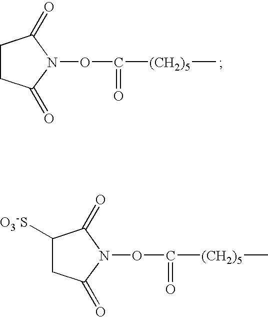

When the complementary group is a thiol, Q^{a} is preferably chosen from:

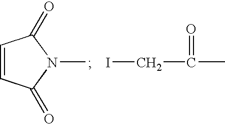

General methods for conjugation of cyanine dyes to biological molecules are described by Licha et al [Topics Curr.Chem., 222, 1-29 (2002); Adv.Drug Deliv.Rev., 57, 1087-1108 (2005)]. Peptide, protein or oligonucleotide BTM for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

Cy^{D} dyes of the present invention may be prepared by a process comprising reaction of:
(a) a first compound having the formula (A):

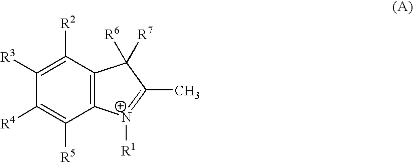

wherein:
   groups R¹ to R⁷ are as defined for the first aspect (above); and
(b) a second compound having the formula (IIa):

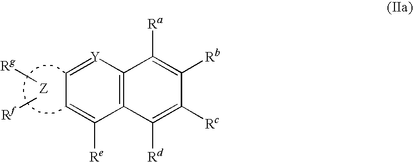

wherein:
groups R^{a} to R^{g}, Y and Z are as defined for the first aspect (above); and
(c) a third compound (C) suitable for forming a conjugated linkage Q between said first and second compounds.

According to the method, intermediate compounds (A), (IIa) and (C) may be reacted in a two step process. In this process, an intermediate compound is first formed by reacting an indolium compound of formula (A) with a compound (C) suitable for forming the linkage, for example, a suitably substituted N,N′-diphenylformamidine, or malonaldehyde dianil, in the presence of acetic anhydride, to form a 2-anilinovinyl or 4-anilino-1,3-butadienyl quaternary salt. The intermediate quaternary salt may be reacted with an aromatic heterocycle such as benzo[b]pyrilium, quinolinium, or an acridinium moiety having a suitably reactive methyl group. Suitably, the reaction is performed in the presence of acetic anhydride and potassium acetate at ambient temperature.

Alternative intermediates for forming the polymethine linkage joining the heterocyclic ring systems are known and are described, for example in Hamer, F. M., “The Cyanine Dyes and Related Compounds”, Interscience (1964). The Cy^{D} dyes of the present invention may optionally be modified to include charged or polar groups may be added to enhance the solubility of the compound in polar or nonpolar solvents. As examples of such modifications, carboxylic acid groups may be converted into esters and amide groups, N-alkylation of quinaldine, lepidine and acridine may be performed using alkyl halides such as methyl iodide, or with butane sultone, or with co-haloalkylcarboxylic acids.

Cy™ is a trademark of GE Healthcare UK Limited.

In a fifth aspect, the present invention provides a method of preparation of the conjugate of the first aspect, which comprises:
(i) mixing the BTM as defined in the first aspect with the Q^{a}-functionalised Cy^{D} of the fourth aspect;
(ii) incubating said Q^{a}-functionalised Cy^{D} with said BTM under conditions suitable for reaction of the Q^{a} group with the BTM, to give the desired conjugate;
(iii) optional separation and/or purification of the conjugate from the reaction mixture of step (ii).

Preferred aspects of the BTM are as described for the first aspect (above).

Covalent labelling of proteins is typically performed in an aqueous buffered medium, suitably bicarbonate at pH 9.0, at ambient temperature for a period of typically 1 hour. The reaction is normally carried out in the dark. The labelled protein can be separated from any unreacted dye by size exclusion chromatography, for example using Sephadex as the stationary phase and phosphate buffer, pH 7.0 as the eluant. For multiple labelling of a BTM, the ratio of the amount or concentration of dye to BTM should be adjusted accordingly.

In addition to the foregoing labelling process, the present invention also relates to two-step labelling processes in which, in a first step, the Q^{a}-functionalised Cy^{D} of the fourth aspect binds to, and thereby labels a primary component, such as an antibody, protein, DNA probe, etc. In the second step of the labelling process, the fluorescently labelled primary component is then used as a probe for detection of a secondary component, such as an antigen for which the antibody is specific.

Preferably, the method of preparation of the conjugate comprises either:
(i) reaction of an amine functional group of a BTM with a compound of formula Y¹-(L)ⱼ-[Cy^{D}]; or
(ii) reaction of a carboxylic acid or activated ester functional group of a BTM with a compound of formula Y²-(L)ⱼ-[Cy^{D}];
(iii) reaction of a thiol group of a BTM with a compound of formula Y³-(L)ⱼ-[Cy^{D}];
   wherein BTM, M^{IG}, L, j and Cy^{D} are as defined above, and
   Y¹ is a carboxylic acid, activated ester, isothiocyanate or thiocyanate group;
   Y² is an amine group;
   Y³ is a maleimide group.

Y² is preferably a primary or secondary amine group, most preferably a primary amine group. In step (iii), the thiol group of the BTM is preferably from a cysteine residue.

In steps (i) to (iii), the BTM may optionally have other functional groups which could potentially react with the Cy^{D} derivative, protected with suitable protecting groups, so that chemical reaction occurs selectively at the desired site only. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl), tert-Butylthio, methoxybenzyl, methylbenzyl or Npys (3-nitro-2-pyridine sulfenyl). The use of further protecting groups are described in ‘Protective Groups in Organic Synthesis’, Theodora W. Greene and Peter G. M. Wuts, (John Wiley & Sons, 1991). Preferred amine protecting groups are Boc and Fmoc, most preferably Boc. Preferred thiol protecting groups are Trt and Acm.

Methods of conjugating dyes to amino acids and peptides are described by Licha (vide supra), as well as Flanagan et al [Bioconj.Chem., 8, 751-756 (1997)]; Lin et al, [ibid, 13, 605-610 (2002)] and Zaheer [Mol.Imaging, 1(4), 354-364 (2002)]. Methods of conjugating the linker group (L) to the BTM employ analogous chemistry to that of the dyes alone (see above), and are known in the art.

The following more specific examples of Cy^{D} dyes of the invention are shown in Tables 2, 3 and 4:

TABLE 2
Trimethine Dyes

| | |
|---|---|
| | (i) |
| 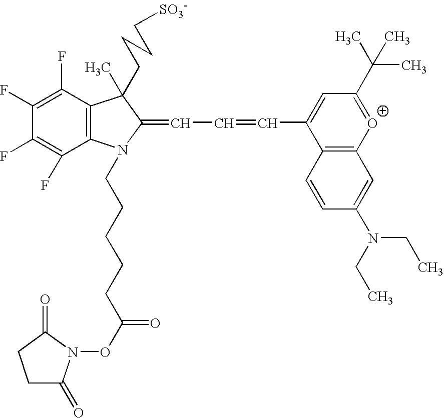 | |
| | (ii) |
| 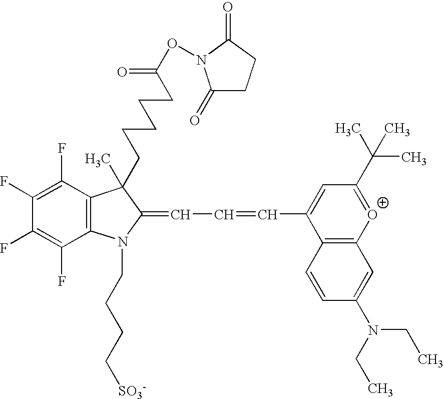 | |

| | |
|---|---|
| | (iii) |
| 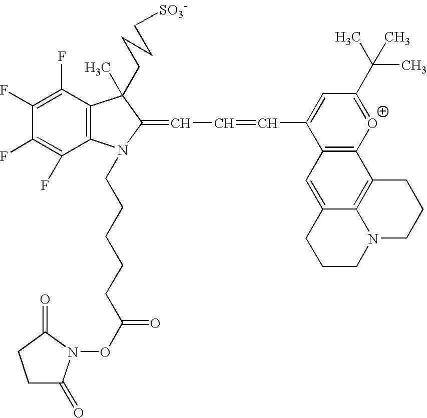 | |
| | (iv) |
| 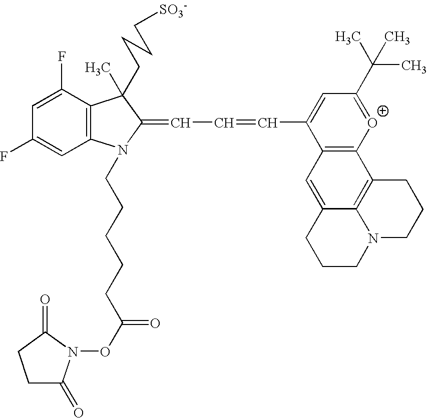 | |

| | |
|---|---|
| | (v) |
| 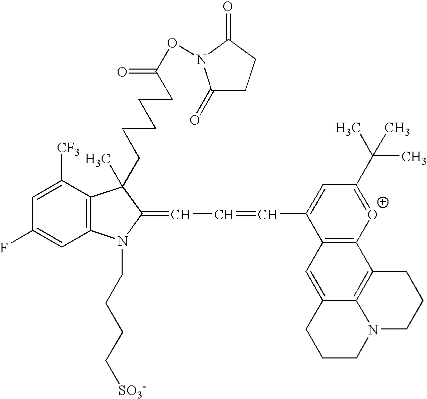 | |

| | |
|---|---|
| | (vi) |
| 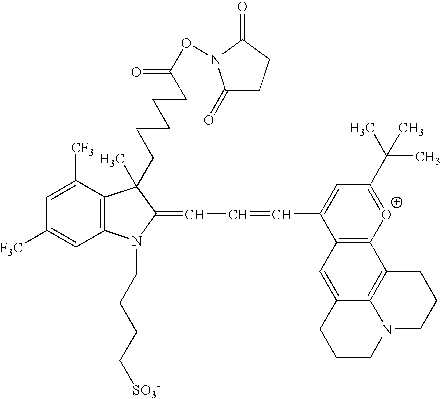 | |
| | (vii) |
| 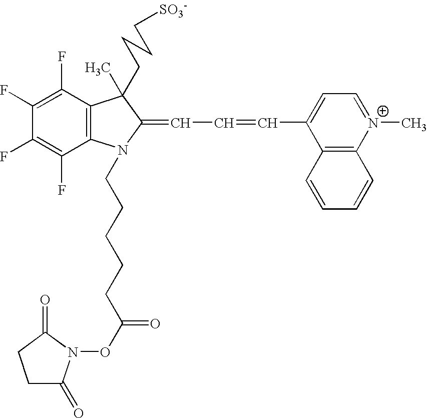 | |
| | (viii) |
| 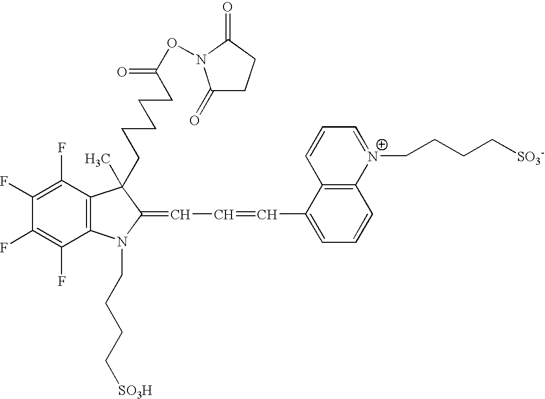 | |
| | (ix) |
| 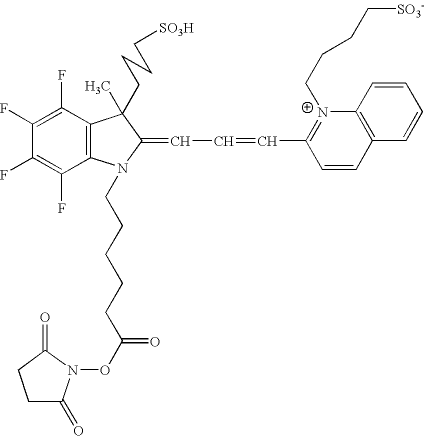 | |

| | |
|---|---|
| | (x) |
| 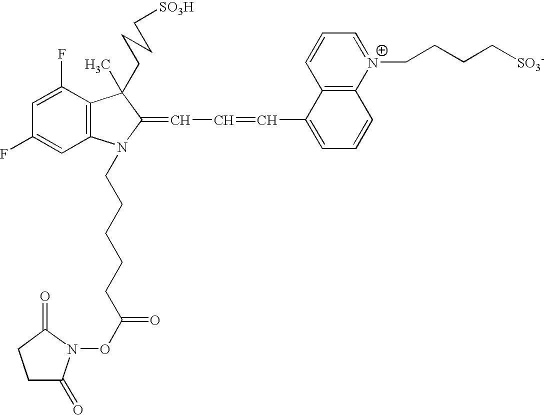 | |

| | |
|---|---|
| | (xi) |
| 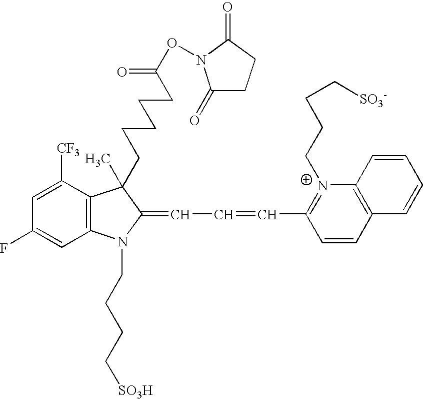 | |
| | (xii) |
| 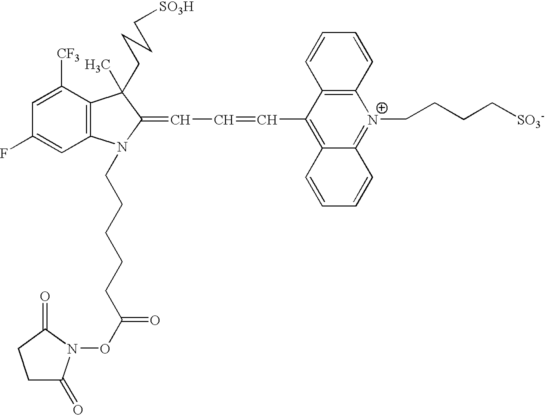 | |

TABLE 3
Pentamethine Dyes.

| | |
|---|---|
| | (xiii) |
| 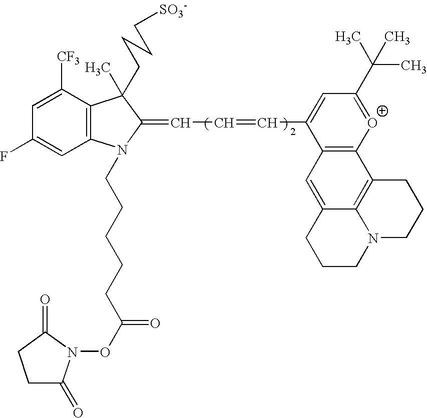 | |
| | (xiv) |
| 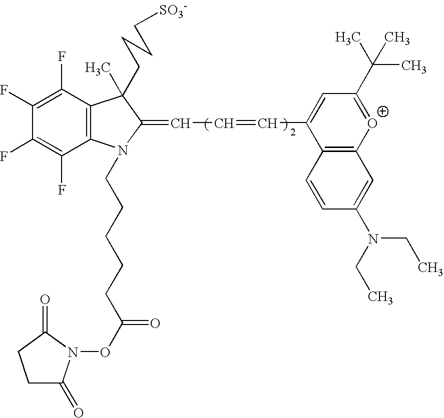 | |
| | (xv) |
| 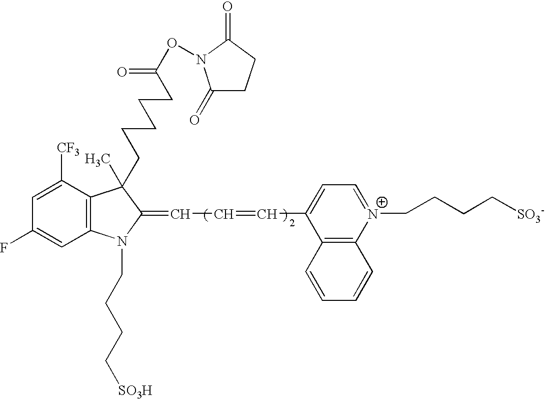 | |
| | (xvi) |
| 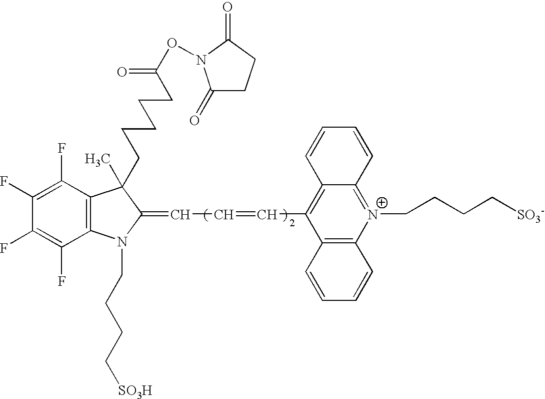 | |
| | (xvii) |
| 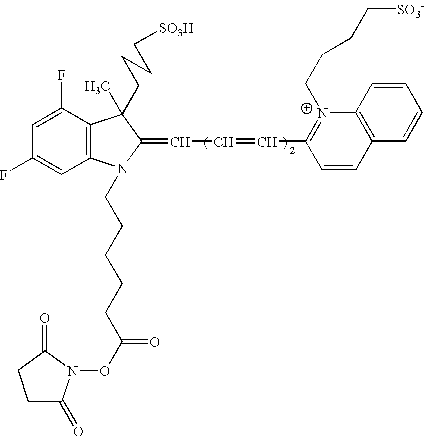 | |

| | |
|---|---|
| | (xviii) |
| 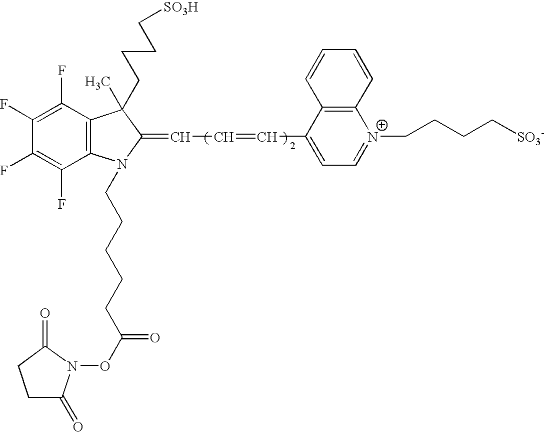 | |

| | |
|---|---|
| | (xix) |
| 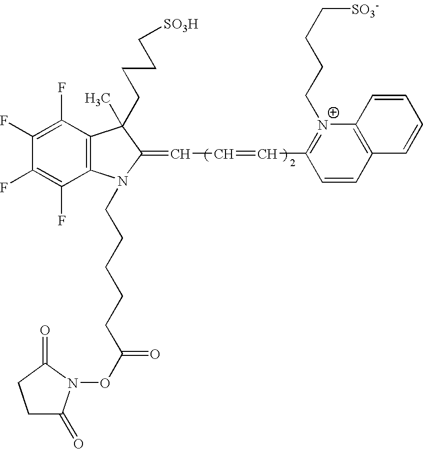 | |
| | (xx) |
| 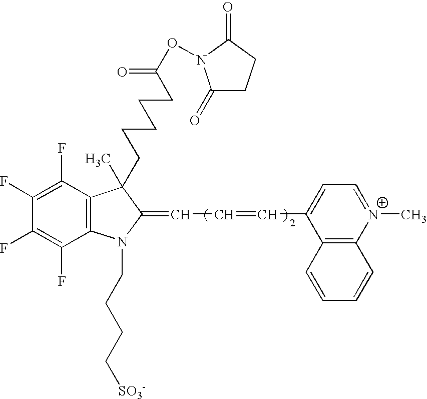 | |

Heptamethine Dyes

| | |
|---|---|
| | (xxi) |
| 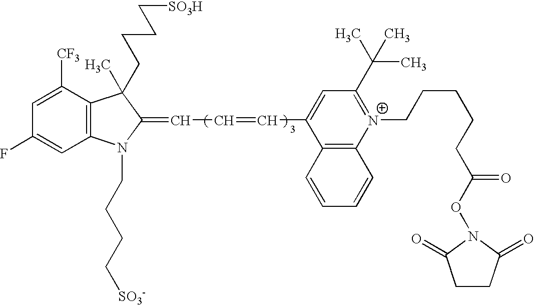 | |
| | (xxii) |
| 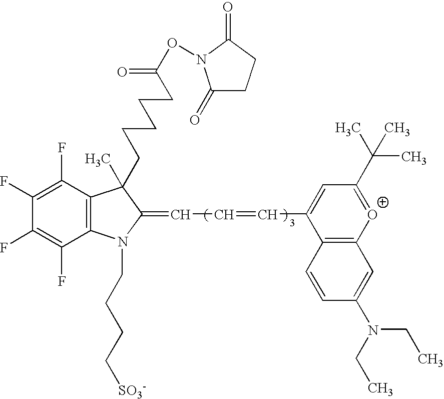 | |

In a sixth aspect, the present invention provides a method of in vivo optical imaging of the mammalian body which comprises use of either the conjugate of the first aspect or the pharmaceutical composition of the second aspect to obtain images of sites of BTM localisation in vivo.

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, e.g. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The green to near-infrared region light is preferably of wavelength 600-1000 nm. The optical imaging method is preferably fluorescence endoscopy. The mammalian body of the sixth aspect is preferably the human body. Preferred embodiments of the conjugate are as described for the first aspect (above). In particular, it is preferred that the Cy^{D} dye employed is fluorescent, photostable, having a Stokes shift of greater than 20 nm, with a high quantum yield, and also is water soluble.

In the method of the sixth aspect, the conjugate or pharmaceutical composition has preferably been previously administered to said mammalian body. By “previously administered” is meant that the step involving the clinician, wherein the agent is given to the patient e.g. as an intravenous injection, has already been carried out prior to imaging. This embodiment includes the use of the conjugate of the first embodiment for the manufacture of a diagnostic agent for the diagnostic imaging in vivo of disease states of the mammalian body where the BTM is implicated.

A preferred optical imaging method of the sixth aspect is Fluorescence Reflectance Imaging (FRI). In FRI, the agent of the present invention is administered to a subject to be diagnosed, and subsequently a tissue surface of the subject is illuminated with an excitation light—usually continuous wave (CW) excitation. The light excites the dye molecule. Fluorescence from the Cy^{D}, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The imaging agent is designed to concentrate in the disease area, producing higher fluorescence intensity. Thus the disease area produces positive contrast in a fluorescence intensity image. The image is preferably obtained using a CCD camera or chip, such that real-time imaging is possible.

The wavelength for excitation varies depending on the particular Cy^{D} dye used. The apparatus for generating the excitation light may be a conventional excitation light source such as: a laser (e.g., ion laser, dye laser or semiconductor laser); halogen light source or xenon light source. Various optical filters may optionally be used to obtain the optimal excitation wavelength.

A preferred FRI method comprises the steps as follows:

(i) a tissue surface of interest within the mammalian body is illuminated with an excitation light;
(ii) fluorescence from the imaging agent, which is generated by excitation of the Cy^{D}, is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

In step (i), the excitation light is preferably continuous wave (CW) in nature. In step (iii), the light detected is preferably filtered. An especially preferred FRI method is fluorescence endoscopy.

An alternative imaging method of the sixth aspect uses FDPM (frequency-domain photon migration). This has advantages over continuous-wave (CW) methods where greater depth of detection of the IM within tissue is important [Sevick-Muraca et al, Curr. Opin. Chem. Biol., 6, 642-650 (2002)]. For such frequency/time domain imaging, it is advantageous if the Cy^{D} has fluorescent properties which can be modulated depending on the tissue depth of the lesion to be imaged, and the type of instrumentation employed.

The FDPM method is as follows:

(a) exposing light-scattering biological tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the imaging agent and preferably further comprises mapping a number of quantities corresponding to adsorption and scattering coefficients of the tissue before administration of the agent. The fluorescence characteristic of step (c) preferably corresponds to at least one of fluorescence lifetime, fluorescence quantum efficiency, fluorescence yield and imaging agent uptake. The fluorescence characteristic is preferably independent of the intensity of the emission and independent of imaging agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The optical imaging of the sixth aspect is preferably used to help facilitate the management of a disease state of the mammalian body. By the term “management” is meant use in the: detection, staging, diagnosis, monitoring of disease progression or the monitoring of treatment. The disease state is suitably one in which the BTM of the imaging agent is implicated. Disease states are preferably located near the body surface or in body cavities or can be exposed by surgical procedures.

Further details of suitable optical imaging methods have been reviewed by Sevick-Muraca et al [Curr.Opin.Chem.Biol., 6, 642-650 (2002)].

In a seventh aspect, the present invention provides a method of detection, staging, diagnosis, monitoring of disease progression or monitoring of treatment of a disease state of the mammalian body which comprises the in vivo optical imaging method of the sixth aspect.

In a further aspect, the present invention provides a method for the assay of an analyte in a sample which method comprises:
(i) contacting the analyte with a specific binding partner for said analyte under conditions suitable to cause the binding of at least a portion of said analyte to said specific binding partner to form a complex, wherein said specific binding partner comprises the dye conjugate of the first aspect;
(ii) measuring the emitted fluorescence of the labelled complex; and
(iii) correlating the emitted fluorescence with the presence or the amount of said analyte in said sample.

In one embodiment, the assay method is a direct assay for the measurement of an analyte in a sample. Optionally, a known or putative inhibitor compound may be included in the assay mix, in which case, the measurement may be correlated with the biological activity of the known or putative inhibitor. In a second, or alternative embodiment, the assay may be a competitive assay, wherein a sample containing an analyte competes with the fluorescent conjugate for a limited number of binding sites on a binding partner that is capable of specifically binding both the analyte and the conjugate. Increasing amounts (or concentrations) of the analyte in the sample will reduce the amount of the fluorescent conjugate of Formula II that is bound to the specific binding partner. The fluorescence signal is measured and the concentration of analyte may be obtained by interpolation from a standard curve.

In a further embodiment, the binding assay may employ a two-step format, wherein a first component, which may be optionally coupled to an insoluble support, is bound to a second component to form a specific binding complex, which is bound in turn to a third component. In this format, the third component is capable of specifically binding to either the second component, or to the specific binding complex. Either of the second or the third component may be the conjugate of the present invention. Examples include “sandwich” assays, in which one component of a specific binding pair, such as a first antibody, is coated onto a surface, such as the wells of a multiwell plate. Following the binding of an antigen to the first antibody, a fluorescent labelled second antibody is added to the assay mix, so as to bind with the antigen-first antibody complex. The fluorescence signal is measured and the concentration of antigen may be obtained by interpolation from a standard curve.

Examples of analyte-specific binding partner pairs include, but are not restricted to, antibodies/antigens, lectins/glycoproteins, biotin/streptavidin, hormone/receptor, enzyme/substrate or co-factor, DNA/DNA, DNA/RNA and DNA/binding protein. It is to be understood that any molecules which possess a specific binding affinity for each other may be employed, so that the fluorescent dyes of the present invention may be used for labelling one component of a specific binding pair, which in turn may be used in the detection of binding to the other component.

The compounds of the present invention may also be used in a detection method wherein a plurality of the fluorescent dyes are covalently attached to a plurality of different primary components, such as antibodies, each primary component being specific for a different secondary component, such as an antigen, in order to identify each of a plurality of secondary components in a mixture of secondary components. According to this method of use, each of the primary components is separately labelled with a fluorescent dye having a different light absorption and emission wavelength characteristic, compared with the dye molecules used for labelling the other primary components. The labelled primary components are then added to the preparation containing secondary components, such as antigens, and the primary components are allowed to attach to the respective secondary components for which they are selective.

Any unreacted probe materials may be removed from the preparation by, for example, washing, to prevent interference with the analysis. The preparation is then subjected to a range of excitation wavelengths including the absorption wavelengths of particular fluorescent compounds. A fluorescence microscope or other fluorescence detection system, such as a flow cytometer or fluorescence spectrophotometer, having filters or monochromators to select the rays of the excitation wavelength and to select the wavelengths of fluorescence is next employed to determined the intensity of the emission wavelengths corresponding to the fluorescent compounds utilized, the intensity of fluorescence indicating the quantity of the secondary component which has been bound with a particular labelled primary component. Known techniques for conducting multi-parameter fluorescence studies include, for example, multiparameter flow cytometry. In certain cases a single wavelength of excitation can be used to excite fluorescence from two or more materials in a mixture where each fluoresces at a different wavelength and the quantity of each labelled species can be measured by detecting its individual fluorescence intensity at its respective emission wavelength. If desired, a light absorption method can also be employed.

The detection method of the present invention can be applied to any system in which the creation of a fluorescent primary component is possible. For example, an appropriately reactive fluorescent compound can be conjugated to a DNA or RNA fragment and the resultant conjugate then caused to bind to a complementary target strand of DNA or RNA. Appropriate fluorescence detection equipment can then be employed to detect the presence of bound fluorescent conjugates.

The invention is further illustrated by reference to the following examples and figures in which:

Examples 1 to 7 provide the syntheses of various fluorinated dye precursors of Formula A of the present invention, which can be reacted with components (IIa) and (C) as described above to give dyes of the invention. Examples 8 and 9 provide [benzo[b]pyrilium compounds of Formula (IIa), suitable for coupling with the precursors of Examples 1 to 7. Example 10 provides a method of N-alkylation of heterocyclic N atoms to introduce 4-sulfobutyl substituents. Examples 11 to 23 provide dyes of Formulae III, IVb and IVc of the invention. Examples 11-18 provide the syntheses of pentamethine dyes of the invention. Examples 19-21 provide the syntheses of trimethine dyes of the invention. Examples 22-23 demonstrate the synthesis of heptamethine dyes of the invention. Examples 24 and 25 provide dyes of the invention substituted with an NHS ester (Q^{a} group). Example 26 provides a photostability study demonstrating that the fluorinated dyes generally demonstrate greater resistance to photobleaching when compared with the non-fluorinated dye analogues. Control samples that were not exposed to light over the same time period, showed no reduction in absorbance. Example 27 provides the synthesis of RGD peptide conjugates of dyes of the invention.

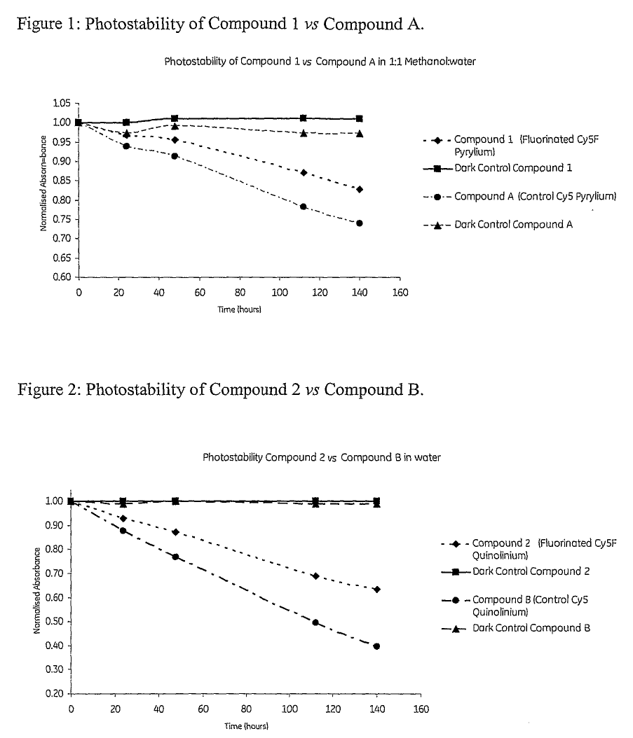

FIGS. 1 and 2 show the improved photostability of dyes of the invention.

### ABBREVIATIONS

DMF: Dimethylformamide
HATU: O-(7-Azabenzotriazol-1-yl)-N,N,N′,N′-tetramethyluronium hexafluorophosphate
HPLC: High performance liquid chromatography
NHS: N-hydroxy-succinimide
NMM: N-methylmorpholine
NMP: 1-Methyl-2-pyrollidine
LC-MS: Liquid chromatography mass spectroscopy
TFA: Trifluoroacetic acid
TLC: Thin layer chromatography.

### Example 1

### 4-[1-(5-carboxypentyl)-4,5,6,7-tetrafluoro-2,3-dimethyl-3H-indolium-3-yl]butane-1-sulfonate

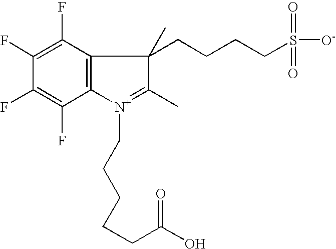

### 1.1 Sodium 5-(ethoxycarbonyl)-5-methyl-6-oxoheptane-1-sulphonate

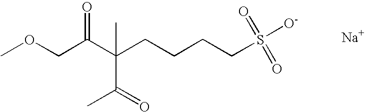

Sodium hydride (60 wt %, 12 g≡0.3 mol NaH) was slurried in dry DMF (100 ml). The resulting suspension was cooled with stirring to 0° C. To this was added a solution of ethyl 2-methylacetoacetate (50 g, 0.346 mol) in DMF (25 ml), dropwise so as to maintain the temperature at <10° C. and control effervescence. Once addition was complete and hydrogen evolution ceased, the mixture was warmed in a warm water bath until a clear, pale yellow solution resulted. This was cooled again to 0° C. A solution of 1,4-butanesultone (45 g, 0.33 mol) in DMF (25 ml) was added over 15 mins, maintaining the temperature at <10° C. Once addition was complete, the mixture was heated at 50° C. for 16 hrs. The solvent was then evaporated under vacuum to dryness; the residue was partitioned between water and diethyl ether. The aqueous layer was retained; the organic layer was extracted with fresh water, then discarded. The combined aqueous extracts were washed with fresh ether, then evaporated under vacuum to give the title product as a waxy solid. δH (270 MHz; D₂O) 4.23 (2H, q), 2.9 (2H, app t), 2.26 (3H, s), 2.0-1.6 (6H, m), 1.36 (3H, s) and 1.26 (3H, t).

### 1.2 5-methyl-6-oxoheptane-1-sulfonic acid

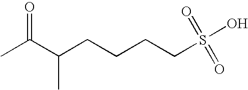

Sodium 5-(ethoxycarbonyl)-5-methyl-6-oxoheptane-1-sulphonate (from 1.1) was heated at 90° C. in concentrated hydrochloric acid (200 ml), until TLC indicated complete reaction (˜3 hrs). The solvent was then evaporated under vacuum; the residue was purified by flash chromatography (Silica. Ethanol/dichloromethane mixtures) to give 49.6 g of the title compound. δH (270 MHz; D₂O) 2.9 (2H, app t), 2.68 (1H, m), 2.2 (3H, s), 1.8-1.3 (6H, m) and 1.18 (3H, d).

### 1.3 2,3-dimethyl-3-(4-sulfonatobutyl)-4,5,6,7-tetrafluoro-3H-indole

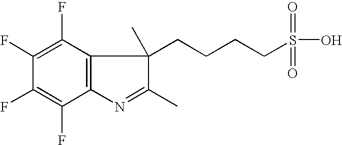

2,3,4,5-Tetrafluoroaniline (1.75 g, 0.01M) was dissolved in conc. HCl (280 ml). The flask was maintained at −10° C. and a solution of NaNO₂ (1 eq) in water (10 ml) added dropwise followed subsequently by a solution of tin(II) chloride (3.4 g) in conc. HCl (40 ml). The reaction was returned to ambient temperature and stirred for 1 hr. The solvent was removed in vacuo to yield crude 2,3,4,5-tetrafluorophenylhydrazine hydrochloride as a yellow solid (7 g). This was redissolved in acetic acid (50 ml) then 5-methyl-6-oxoheptane-1-sulfonic acid (6 g) was added. The solution was heated at 140° C. for 2 hrs to yield an orange solution with fine orange precipitate. The solvent was evaporated to yield a brown gum, from which the title product was isolated by reverse phase HPLC (0.1% TFA, water/acetonitrile gradient). MS (MALDI-TOF), MH⁺=354.

### 1.4 4-[1-(5-carboxypentyl)-4,5,6,7-tetrafluoro-2,3-dimethyl-3H-indolium-3-yl]butane-1-sulfonate

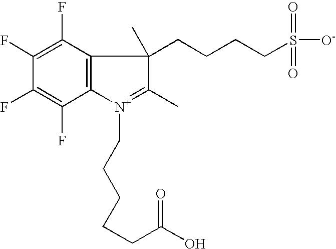

Tetra-fluorinated indole (from 1.3) (150 mg, 4.2×10⁻⁴ mol, 1 eq.) was heated at 140° C. with 6-bromohexanoic acid (15 g, 0.073 mol, 260 eq) for 24 hr under nitrogen. The final reaction mixture was triturated with diethyl ether and dried under vacuum to yield a brown mass. The major component was confirmed as the title compound by LC-MS. MS (MALDI-TOF), MH⁺=470.

### Example 2

### 4-[3-(5-carboxypentyl)-4,5,6,7-tetrafluoro-2,3-dimethyl-3H-indolium-1-yl]butane-1-sulfonate

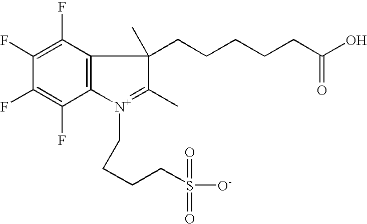

### 2.1 6-(4,5,6,7-tetrafluoro-2,3-dimethyl-3H-indol-3-yl)hexanoic acid

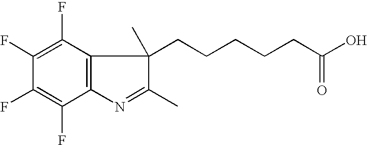

To 2,3,4,5-tetrafluorophenylhydrazine hydrochloride (2 g) was added 7-methyl-8-oxononanoic acid (3 g) and acetic acid (50 ml) and the mixture heated to 140° C. for 5 hours. On cooling the volatile components were removed on a rotary evaporator and the residue dissolved in water (10 ml), filtered and purified by preparative HPLC in 2 shots to give the title compound. MS (MALDI-TOF), MH⁺=332

### 2.2 4-[3-(5-carboxypentyl)-4,5,6,7-tetrafluoro-2,3-dimethyl-3H-indolium-1-yl]butane-1-sulfonate

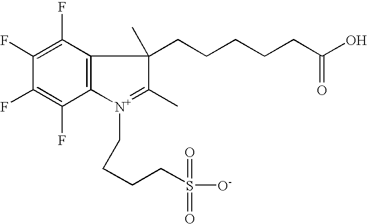

To 6-(4,5,6,7-tetrafluoro-2,3-dimethyl-3H-indol-3-yl)hexanoic acid (600 mg) was added butane sultone (4 ml) and the mixture heated to 140° C. overnight. On cooling the mixture was diluted with water (4 ml), filtered and purified by preparative HPLC to give the title compound, 800 mg. MS (MALDI-TOF), MH⁺=468

### Example 3

### 4-[1-(5-carboxypentyl)-4,6-difluoro-2,3-dimethyl-3H-indolium-3-yl]butane-1-sulfonate

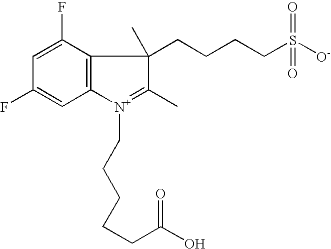

### 3.1 4,6-Difluoro-2,3-dimethyl-3-(4-sulfonatobutyl)-3H-indole

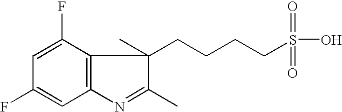

To 3,5-difluorophenylhydrazine hydrochloride (1 g) in acetic acid (20 ml) was added 5-methyl-6-oxoheptane-1-sulfonic acid (1.6 g) and the solution heated to reflux overnight. The volatiles were removed on a rotary evaporator to give the crude product, 50 mg of which was purified by preparative HPLC. The relevant fractions were combined, concentrated on a rotary evaporator and freeze-dried to give the title product, (19 mg). MS (MALDI-TOF), MH⁺=317.

### 3.2 4-[1-(5-carboxypentyl)-4,6-difluoro-2,3-dimethyl-3H-indolium-3-yl]butane-1-sulfonate

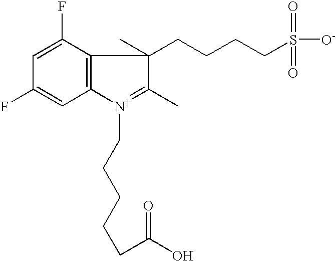

To 4,6-difluoro-2,3-dimethyl-3-(4-sulfonatobutyl)-3H-indole (0.8 g of non purified material) was added 6-bromohexanoic acid (1.6 g) and the solution heated to 140° C. for 2 days. On cooling, the product was diluted with acetonitrile and purified by preparative HPLC. The relevant fractions were combined, concentrated on a rotary evaporator and freeze dried to give the desired product (110 mg). MS (MALDI-TOF), MH⁺=432.

### Example 4

### 4-[3-(5-carboxypentyl)-2,3-dimethyl-4,6-bis(trifluoromethyl)-3H-indolium-1-yl]butane-1-sulfonate

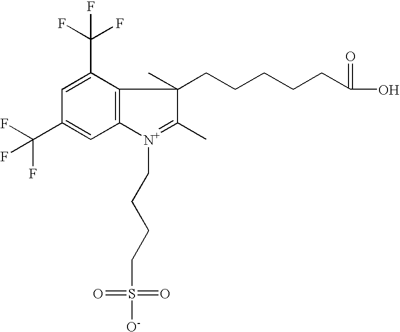

### 4.1 4,6-Bis(trifluoromethyl)-2,3-dimethyl-3-(5-carboxypentyl)-3H-indole

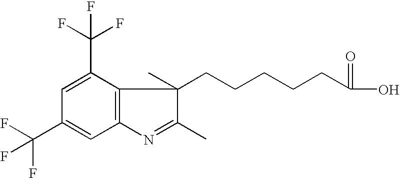

To 3,5-bis(trifluoromethyl)phenylhydrazine hydrochloride(1 g) in acetic acid (20 ml) was added 7-methyl-8-oxononanoic acid (0.66 g) and the solution heated to reflux for 5 hours. The volatiles were removed on a rotary evaporator to give the crude product, 1 ml of which was purified by preparative HPLC. The relevant fractions were combined, concentrated on a rotary evaporator and freeze dried to give the desired product, (17 mg). MS (MALDI-TOF), MH⁺=395.

4.2 To 4,6-bis(trifluoromethyl)-2,3-dimethyl-3-(5-carboxypentyl)-3H-indole (300 mg of non purified material) was added butane sultone (3 ml) and the solution heated to 140° C. for 1 day. On cooling, the product was extracted into water and purified by preparative HPLC. The relevant fractions were combined, concentrated on a rotary evaporator and freeze dried to give the desired product (19 mg). MS (MALDI-TOF), MH⁺=532.

### Example 5

### 4-[3-(5-carboxypentyl)-6-fluoro-2,3-dimethyl-4-(trifluoromethyl)-3H-indolium-1-yl]butane-1-sulfonate

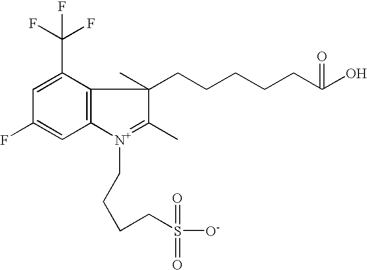

### 5.1 6-(4-trifluoromethyl-6-fluoro-2,3-dimethyl-3H-indol-3-yl)hexanoic acid

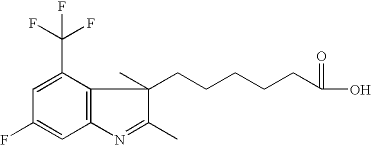

To 3-trifluoromethyl-5-fluorophenyl hydrazine (2 g) was added 7-methyl-8-oxononanoic acid (3 g) and acetic acid (50 ml) and the mixture heated to 140° C. for 5 hours. On cooling the volatile components were removed on a rotary evaporator and the residue dissolved in water (10 ml), filtered and purified by preparative HPLC to give the desired product (600 mg). MS (MALDI-TOF), MH⁺=346

### 5.2 4-[3-(5-carboxypentyl)-6-fluoro-2,3-dimethyl-4-(trifluoromethyl)-3H-indolium-1-yl]butane-1-sulfonate

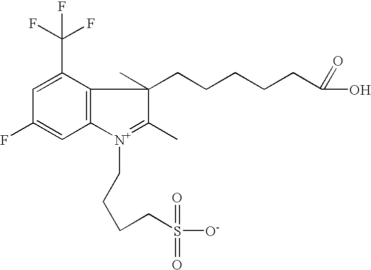

To 6-(4-trifluoromethyl-6-fluoro-2,3-dimethyl-3H-indol-3-yl)hexanoic acid (600 mg) was added 1,4-butane sultone (4 ml) and the mixture heated to 140° C. overnight. On cooling the mixture was diluted with water (4 ml), filtered and purified by preparative HPLC to give the desired product (800 mg). MS (MALDI-TOF), MH⁺=483.

### Example 6

### 4-[1-(5-carboxypentyl)-6-fluoro-2,3-dimethyl-4-(trifluoromethyl)-3H-indolium-3-yl]butane-1-sulfonate

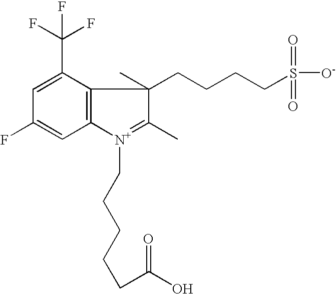

### 6.1 4-trifluoromethyl-6-fluoro-2,3-dimethyl-3-(4-sulfobutyl)-3H-indole

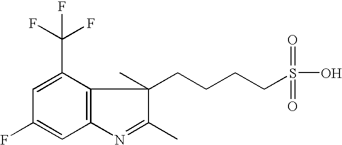

To 3-trifluoromethyl-5-fluorophenylhydrazine (2 g) was added 5-methyl-6-oxoheptane-1-sulfonic acid (3 g) and acetic acid (60 ml) and the mixture heated to 140° C. for 5 hours. After this time the volatiles were removed on a rotary evaporator and the residue dissolved in water (10 ml), filtered and purified by preparative HPLC to give the desired product (790 mg). MS (MALDI-TOF), MH⁺=368.

### 6.2 4-[1-(5-carboxypentyl)-6-fluoro-2,3-dimethyl-4-(trifluoromethyl)-3H-indolium-3-yl]butane-1-sulfonate

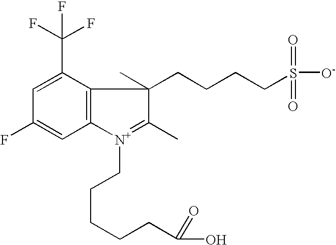

To 4-trifluoromethyl-6-fluoro-2,3-dimethyl-3-(4-sulfobutyl)-3H-indole (790 mg) was added butane sultone (10 ml) and the mixture heated at 140° C. overnight. The mixture was diluted with water (4 ml), filtered and purified by preparative HPLC to give the desired product (1 g). MS (MALDI-TOF), MH⁺=505

### Example 7

### 4-[1-(5-carboxypentyl)-5,7-difluoro-2,3-dimethyl-3H-indolium-3-yl]butane-1-sulfonate

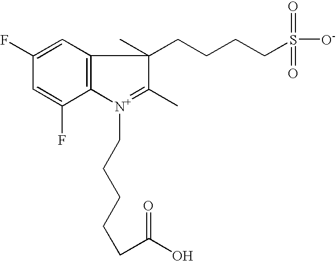

### 7.1 5,7-difluoro-2,3-dimethyl-3-(4-sulfobutyl)-3H-indole

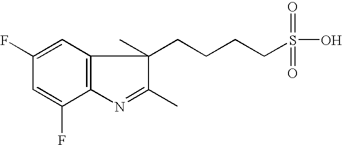

To 2,4-difluorophenyl hydrazine hydrochloride (2 g) in acetic acid (60 ml) was added 5-methyl-6-oxoheptane-1-sulfonic acid (4.5 g) and the solution heated to reflux for 2 hrs. The volatiles were removed on a rotary evaporator to give the crude product, which was purified by flash chromatography (RP-18 silica, water/acetonitrile mixtures as eluent). The relevant fractions (identified by LC-MS) were combined, concentrated on a rotary evaporator and freeze-dried to give the desired product (6 g). MS (MALDI-TOF), MH⁺=317.

### 7.2 4-[1-(5-carboxypentyl)-5,7-difluoro-2,3-dimethyl-3H-indolium-3-yl]butane-1-sulfonate

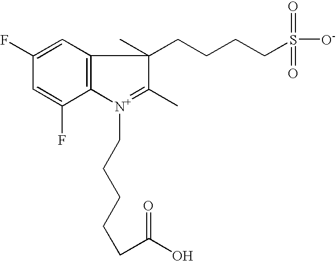

To 5,7-difluoro-2,3-dimethyl-3-(4-sulfobutyl)-3H-indole (1.0 g) was added 6-bromohexanoic acid (5 g) and the solution heated to 140° C. for 2 days. On cooling, the product was triturated with diethyl ether to give a slurry. The solids were collected by filtration, washed with ether and dried under vacuum to give the crude product. This was further purified by preparative HPLC to give the title product (300 mg). MS (MALDI-TOF), MH⁺=432.

### Example 8

### 2-tert-Butyl-7-dimethylamino-4-methylchromenylium tetrafluoroborate

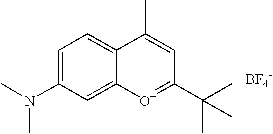

### 8.1 2-tert-Butyl-7-(dimethylamino)-4H-chromen-4-one

The preparation of this class of compound is described in WO 92/09661 (Telfer, et al), Example 1. According to this method, 3-dimethylaminophenol (4.4 g, 0.032 mol) and methyl 4,4-dimethyl-3-oxopentanoate (8.8 g, 0.056 mol) were stirred at 180° C. (Woods metal bath) under nitrogen for 40 hours. The reaction mixture was cooled, dissolved in dichloromethane/hexane and purified by silica gel flash chromatography using ethyl acetate/hexane mixtures. Yield 5.41 g. MS (MALDI-TOF), MH⁺=246. UV/VIS (MeOH): 347, 294, 263 and 215 nm.

### 8.2 2-tert-Butyl-7-dimethylamino-4-methylchromenylium-tetrafluoroborate

The preparation of this compound is described in WO 92/09661 (Telfer, et al), Example 3. Methyl magnesium bromide (4.5 ml of a 3M solution in ether, 13.5.5 mmol) was added drop-wise to a solution of 2-tert-Butyl-7-(dimethylamino)-4H-chromen-4-one (2.45 g, 10 mmol) in dry tetrahydrofuran (24 ml) at 0° C. under nitrogen. The reaction mixture was stirred at 25° C. for 17 hours and then poured into ice water. Tetrafluoroboric acid (48 wt % aq., 5 ml) was added and the mixture stirred briefly, then extracted with dichloromethane (100 ml+2×25 ml). The organic extracts were combined dried (MgSO₄), filtered and evaporated under vacuum to low volume. Ethyl acetate (100 ml) was added and evaporation continued, giving the product as a yellow solid. This was slurried in more ethyl acetate, collected by filtration and dried under vacuum. Yield 2.97 g. MS (MALDI-TOF), MH⁺=244. UV/VIS (MeOH): 468, 287 and 219 nm.

### Example 9

### 11-tert-butyl-9-methyl-2,3,6,7-tetrahydro-1H,5H-pyrano[2,3-f]pyrido[3,2,1-ij]quinolin-12-ium tetrafluoroborate

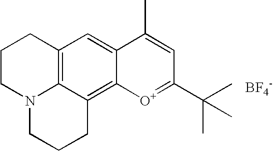

This was prepared in a similar manner to that given in Example 8, except that the 3-dimethylaminophenol starting material was replaced with 8-hydroxyjulolidine. MS (MALDI-TOF), MH⁺=296. UV/VIS (MeOH): 486, 359, 299, 275 and 228 nm.

### Example 10

### General Method for N-alkylation of Quinaldine, Lepidine and Acridine with Butane Sultone

N-Alkylation of quinaldines, lepidines and 9-methyl-acridines with butane sultone to form N-(4-sulfonatobutyl)-derivatives may be performed by a method analogous to that described in U.S. Pat. No. 6,579,718 (Yue et al). Typically, a mixture of the nitrogen heteroaromatic and butane sultone (excess) are heated at up to 140° C. for up to 24 hours whilst stirring. After cooling to room temperature the mixture is triturated with ethyl acetate or diethyl ether to yield the N-alkylated product, which is used directly in dye synthesis. The following example describes a typical synthesis.

### 4-(4-Methylquinolinium-1-yl)butane-1-sulfonate

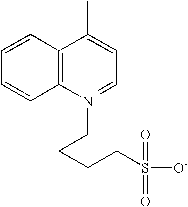

A mixture of lepidine (2.86 g) and 1,4-butane sultone (5 ml) were mixed and heated at 65° C. for 17 hrs, during which time a white solid separated. After allowing the mixture to cool to ambient temperature, the mixture was diluted with ether and the solid collected by filtration, washed with fresh ether and dried under vacuum to give the title compound, 3.14 g. MS (MALDI-TOF), MH⁺=280.

### Example 11

### 4-[(4E)-4-{(2E,4E)-5-[1-(5-carboxypentyl)-4,5,6,7-tetrafluoro-3-methyl-3-(4-sulfobutyl)-3H-indolium-2-yl]penta-2,4-dienylidene}quinolin-1(4H)-yl]butane-1-sulfonate

### 11.1 4-[2-{(1E,3E)-4-[acetyl(phenyl)amino]buta-1,3-dienyl}-1-(5-carboxypentyl)-4,5,6,7-tetrafluoro-3-methyl-3H-indolium-3-yl]butane-1-sulfonate

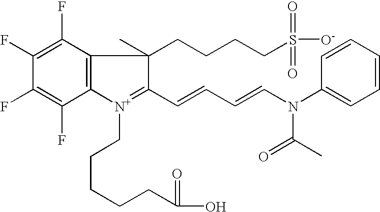

4-[1-(5-carboxypentyl)-4,5,6,7-tetrafluoro-2,3-dimethyl-3H-indolium-3-yl]butane-1-sulfonate (example 1, 1.5 g) was mixed with malonaldehyde bis(phenylimine).HCl (1.0 g) in acetic anhydride (33 ml) and acetic acid (17 ml) and heated for 17 hrs at 120° C. The resulting deep yellow solution was then used directly to make pentamethine dye examples.

### 11.2 4-[(4E)-4-{(2E,4E)-5-[1-(5-carboxypentyl)-4,5,6,7-tetrafluoro-3-methyl-3-(4-sulfobutyl)-3H-indolium-2-yl]penta-2,4-dienylidene}quinolin-1(4H)-yl]butane-1-sulfonate

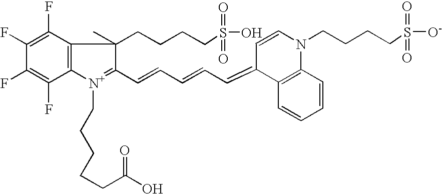

To a flask containing 4-(4-Methylquinolinium-1-yl)butane-1-sulfonate (Example 10, 150 mg) and potassium acetate (1.0 g) was added an aliquot of the solution from example 11.1 (5.0 ml). The resulting mixture was stirred at ambient temperature for 24 hrs, before being added to 100 ml of stirring ethyl acetate. The precipitated solids were collected by filtration, washed with ethyl acetate and ether and air dried. The crude solid was then purified by preparative HPLC to give the title dye. MS (MALDI-TOF), M⁺=783. Accurate mass: C₃₇H₄₃N₂F₄O₈S₂⁺ requires 783.2397, found M⁺@783.2408 (1.4 ppm). UV/VIS(MeOH): 607 nm. Fluorescence (MeOH): excitation λmax=608 nm; emission λmax=724 nm.

### Example 12

### 4-[(2E)-2-{(2E,4E)-5-[1-(5-carboxypentyl)-4,5,6,7-tetrafluoro-3-methyl-3-(4-sulfobutyl)-3H-indolium-2-yl]penta-2,4-dienylidene}quinolin-1(2H)-yl]butane-1-sulfonate (Compound 2)

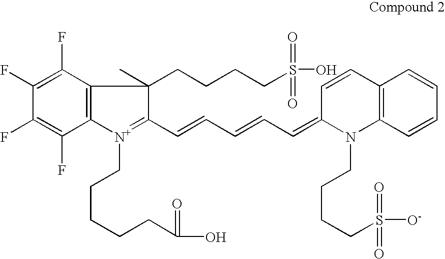

This was prepared as in Example 11.2, except that 4-(2-methylquinolinium-1-yl)butane-1-sulfonate (150 mg) was used. MS (MALDI-TOF), M⁺=783. Accurate mass: C₃₇H₄₃N₂F₄O₈S₂⁺ requires 783.2397, found M⁺@783.2419 (2.8 ppm). UV/VIS(MeOH): 598 nm. Fluorescence (MeOH): excitation λmax=600 nm; emission λmax=680 nm.

### Example 13

### 4-[(2E)-2-{(2E,4E)-5-[1-(5-carboxypentyl)-4,5,6,7-tetrafluoro-3-methyl-3-(4-sulfobutyl)-3H-indolium-2-yl]penta-2,4-dienylidene}-6-methylquinolin-1(2H)-yl]butane-1-sulfonate

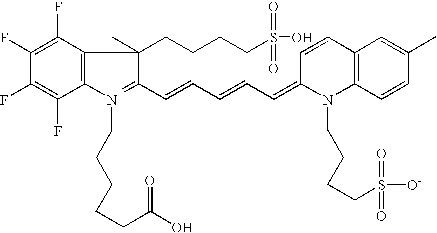

This was prepared as in example 11.2, except that 4-(2,6-dimethylquinolinium-1-yl)butane-1-sulfonate (150 mg) was used. MS (MALDI-TOF), M⁺=797. Accurate mass: C₃₈H₄₅N₂F₄O₈S₂⁺ requires 797.2553, found M⁺@797.2588 (4.3 ppm). UV/VIS(MeOH): 586 nm. Fluorescence (MeOH): excitation λmax=598 nm; emission λmax=678 nm.

### Example 14

### 4-[(2E)-2-{(2E,4E)-5-[1-(5-carboxypentyl)-4,5,6,7-tetrafluoro-3-methyl-3-(4-sulfobutyl)-3H-indolium-2-yl]penta-2,4-dienylidene}-6-fluoroquinolin-1(2H)-yl]butane-1-sulfonate

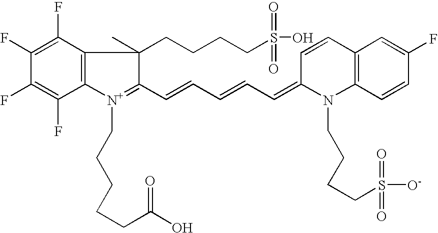

This was prepared as in Example 11.2, except that 4-(2-methyl-6-fluoroquinolinium-1-yl)butane-1-sulfonate (60 mg) was used, with potassium acetate (400 mg) and the solution from example 11.1 (2.0 ml). MS (MALDI-TOF), M⁺=801. Accurate mass: C₃₇H₄₂N₂F₅O₈S₂⁺ requires 801.2303, found M⁺@801.2321 (2.3 ppm). UV/VIS(MeOH): 607 nm. Fluorescence (MeOH): excitation λmax=616 nm; emission ?max=683 nm.

### Example 15

### 4-[(2E)-2-{(2E,4E)-5-[1-(5-carboxypentyl)-4,5,6,7-tetrafluoro-3-methyl-3-(4-sulfobutyl)-3H-indolium-2-yl]penta-2,4-dienylidene}-7-fluoroquinolin-1(2H)-yl]butane-1-sulfonate

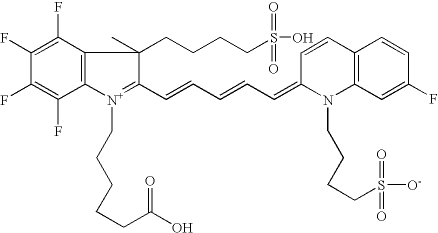

This was prepared as in Example 11.2, except that 4-(2-methyl-7-fluoroquinolinium-1-yl)butane-1-sulfonate (60 mg) (60 mg) was used, with potassium acetate (400 mg) and the solution from example 11.1 (2.0 ml). MS (MALDI-TOF), M⁺=801. Accurate mass: C₃₇H₄₂N₂F₅O₈S₂⁺ requires 801.2303, found M⁺@801.2318 (1.9 ppm). UV/VIS(MeOH): 614 nm. Fluorescence (MeOH): excitation λmax=616 nm; emission λmax=685 nm.

### Example 16

### 4-[(2E)-2-{(2E,4E)-5-[1-(5-carboxypentyl)-4,5,6,7-tetrafluoro-3-methyl-3-(4-sulfobutyl)-3H-indolium-2-yl]penta-2,4-dienylidene}-7-chloroquinolin-1(2H)-yl]butane-1-sulfonate

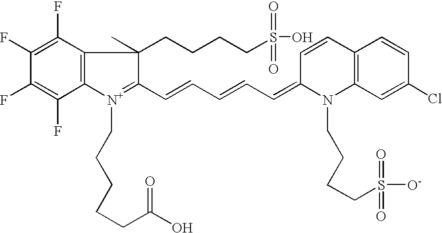

This was prepared as in Example 11.2, except that 4-(2-methyl-7-chloroquinolinium-1-yl)butane-1-sulfonate (60 mg) was used, with potassium acetate (400 mg) and the solution from example 11.1 (2.0 ml). MS (MALDI-TOF), M⁺=817/819. Accurate mass: C₃₇H₄₂N₂ClF₄N₂O₈S₂⁺ (with ³⁵Cl) requires 817.2007, found M⁺@817.2039 (3.9 ppm). UV/VIS(MeOH): 627 nm.

### Example 17

### 4-[2-{(1E,3E,5E)-5-[2-tert-butyl-7-(dimethylamino)-4H-chromen-4-ylidene]penta-1,3-dienyl}-1-(5-carboxypentyl)-4,5,6,7-tetrafluoro-3-methyl-3H-indolium-3-yl]butane-1-sulfonate (Compound 1)

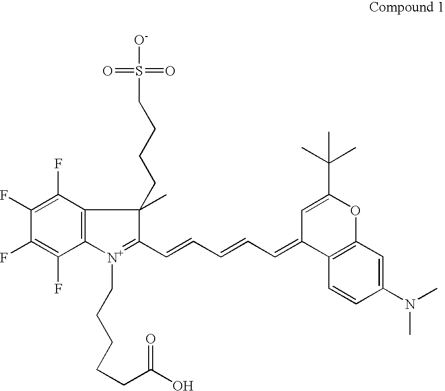

This was prepared as in Example 11.2, except that 2-tert-butyl-7-dimethylamino-4-methylchromenylium tetrafluoroborate (example 8.2, 165 mg) was used. MS (MALDI-TOF), M⁺=747. Accurate mass: C₃₉H₄₇N₂F₄O₆S⁺ requires 747.3091, found M⁺@747.3055 (4.8 ppm). UV/VIS(MeOH): 702 nm. Fluorescence (MeOH): excitation λmax=701 nm; emission λmax=736 nm.

### Example 18

### 4-[2-[(1E,3E5E)-5-(11-tert-butyl-2,3,6,7-tetrahydro-1H,5H,9H-pyrano[2,3-f]pyrido[3,2,1-ij]quinolin-9-ylidene)penta-1,3-dienyl]-1-(5-carboxypentyl)-4,5,6,7-tetrafluoro-3-methyl-3H-indolium-3-yl]butane-1-sulfonate

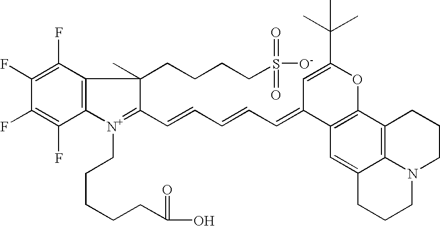

This was prepared as in Example 11.2, except that 11-tert-butyl-9-methyl-2,3,6,7-tetrahydro-1H,5H-pyrano[2,3-f]pyrido[3,2,1-ij]quinolin-12-ium tetrafluoroborate (Example 9, 190 mg) was used. MS (MALDI-TOF), M⁺=799. Accurate mass: C₄₃H₅₁N₂F₄O₆S⁺ requires 799.3404, found M⁺@799.3412 (1.0 ppm). UV/VIS(MeOH): 699 nm. Fluorescence (MeOH): excitation λmax=698 nm; emission λmax=748 nm.

### Example 19

### 4-[2-{(1E,3E)-3-[2-tert-butyl-7-(dimethylamino)-4H-chromen-4-ylidene]prop-1-enyl}-1-(5-carboxypentyl)-4,5,6,7-tetrafluoro-3-methyl-3H-indolium-3-yl]butane-1-sulfonate (Compound 3)

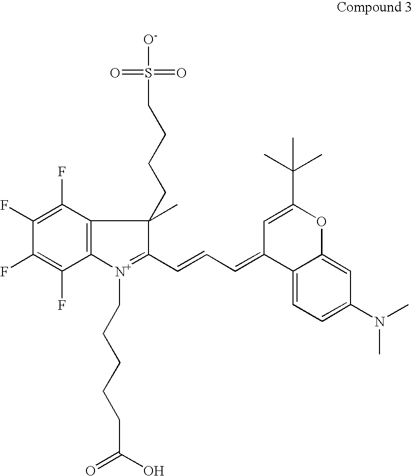

A mixture of 4-[1-(5-carboxypentyl)-4,5,6,7-tetrafluoro-2,3-dimethyl-3H-indolium-3-yl]butane-1-sulfonate (Example 1, 60 mg), 2-tert-butyl-7-dimethylamino-4-methylchromenylium tetrafluoroborate (example 8, 80 mg), triethyl orthoformate (0.5 ml) and pyridine (11.0 ml) were mixed and heated at 120° C. for 3 hrs. The solvent was then evaporated under vacuum and the residue subjected to preparative HPLC, collecting the blue dye fraction. A sample was obtained analytically pure by further preparative TLC (silica. Methanol, 15: ethyl acetate, 85). MS (MALDI-TOF), M⁺=721. Accurate mass: C₃₇H₄₅N₂F₄O₆S⁺ requires 721.2934, found M⁺@721.2946 (1.6 ppm). UV/VIS(MeOH): 617 nm. Fluorescence (MeOH): excitation λmax=616 nm; emission λmax=641 nm.

### Example 20

### 4-[2-{(1E,3E)-3-[2-tert-butyl-7-(dimethylamino)-4H-chromen-4-ylidene]prop-1-enyl}-1-(5-carboxypentyl)-5,7-difluoro-3-methyl-3H-indolium-3-yl]butane-1-sulfonate

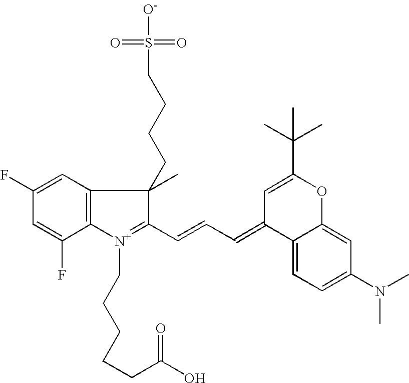

A mixture of 4-[1-(5-carboxypentyl)-5,7-difluoro-2,3-dimethyl-3H-indolium-3-yl]butane-1-sulfonate (Example 7, 100 mg), 2-tert-butyl-7-dimethylamino-4-methylchromenylium tetrafluoroborate (example 8, 100 mg), triethyl orthoformate (0.5 ml) and pyridine (11.0 ml) were mixed and heated at 120° C. for 3 hrs. The solvent was then evaporated under vacuum and the residue subjected to preparative RP-HPLC, collecting the blue dye fraction. A sample was obtained analytically pure by further preparative TLC (silica. Methanol, 15: ethyl acetate, 85). MS (MALDI-TOF), M⁺=685. Accurate mass: C₃₇H₄₇N₂F₂O₆S⁺ requires 685.3123, found M⁺@685.3109 (2.0 ppm). UV/VIS(MeOH): 626 nm. Fluorescence (MeOH): excitation λmax=627 nm; emission λmax=647 nm.

### Example 21

### 4-[2-[(1E,3E)-3-(11-tert-butyl-2,3,6,7-tetrahydro-1H,5H,9H-pyrano[2,3-f]pyrido[3,2,1-ij]quinolin-9-ylidene)prop-1-enyl]-1-(5-carboxypentyl)-5,7-difluoro-3-methyl-3H-indolium-3-yl]butane-1-sulfonate

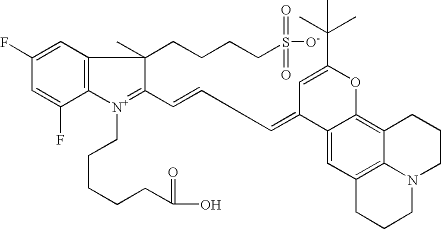

A mixture of 4-[1-(5-carboxypentyl)-5,7-difluoro-2,3-dimethyl-3H-indolium-3-yl]butane-1-sulfonate (Example 7, 100 mg), 11-tert-butyl-9-methyl-2,3,6,7-tetrahydro-1H,5H-pyrano[2,3-f]pyrido[3,2,1-ij]quinolin-12-ium tetrafluoroborate (example 9, 120 mg), triethyl orthoformate (11.0 ml) and pyridine (2.0 ml) were mixed and heated at 120° C. for 3 hrs. The solvent was then evaporated under vacuum and the residue subjected to preparative HPLC, collecting the blue dye fraction. A sample was obtained analytically pure by further preparative TLC (silica. Methanol, 15: ethyl acetate, 85). MS (MALDI-TOF), M⁺=737. Accurate mass: C₄₁H₅₁N₂F₂O₆S⁺ requires 737.3436, found M⁺@737.3433 (0.4 ppm). UV/VIS(MeOH): 639 nm. Fluorescence (MeOH): excitation λmax=639 nm; emission λmax=663 nm.

### Example 22

### 4-[(2E)-2-{(2E,4E,6E)-7-[1-(5-carboxypentyl)-5,7-difluoro-3-methyl-3-(4-sulfobutyl)-3H-indolium-2-yl]hepta-2,4,6-trienylidene}-6-methylquinolin-1(2H)-yl]butane-1-sulfonate

### 22.1 4-[2-{(1E,3E,5E)-6-[acetyl(phenyl)amino]hexa-1,3,5-trienyl}-1-(5-carboxypentyl)-5,7-difluoro-3-methyl-3H-indolium-3-yl]butane-1-sulfonate

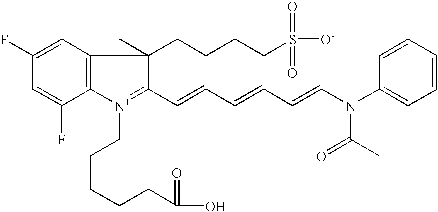

4-[1-(5-carboxypentyl)-5,7-difluoro-2,3-dimethyl-3H-indolium-3-yl]butane 1-sulfonate (example 7, 0.3 g) was mixed with glutaconaldehyde bis(phenylimine).HCl (200 mg) in acetic anhydride (6.7 ml) and acetic acid (3.4 ml) and heated for 17 hrs at 120° C. The resulting deep red solution was then used directly to make heptamethine dye examples.

### 22.2 4-[(2E)-2-{(2E,4E,6E)-7-[1-(5-carboxypentyl)-5,7-difluoro-3-methyl-3-(4-sulfobutyl)-3H-indolium-2-yl]hepta-2,4,6-trienylidene}-6-methylquinolin-1(2H)-yl]butane-1-sulfonate

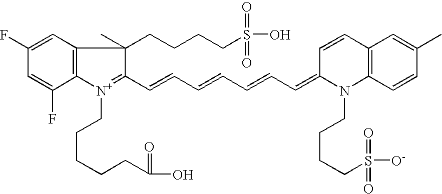

To 2 ml of the heptamethine half-dye solution from 21.1 were added 4-(2,6-dimethylquinolinium-1-yl)butane-1-sulfonate (60 mg) and potassium acetate (400 mg). The resulting mixture was stirred overnight at ambient temperature before being precipitated into excess ethyl acetate. The crude solid dye was collected by filtration, redissolved in water/acetonitrile and purified by preparative RP-HPLC to give the title dye. MS (MALDI-TOF), M⁺=787. UV/VIS(MeOH): 661 nm. Fluorescence (MeOH): emission λmax=800 nm.

### Example 23

### 4-[2-{(1E,3E,5E,7E)-7-[2-tert-butyl-7-(dimethylamino)-4H-chromen-4-ylidene]hepta-1,3,5-trienyl}-1-(5-carboxypentyl)-5,7-difluoro-3-methyl-3H-indolium-3-yl]butane-1-sulfonate

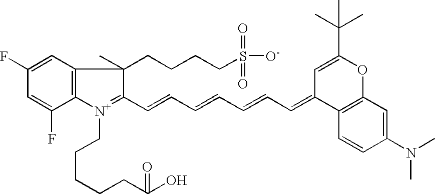

To 2 ml of the heptamethine half-dye solution from 21.1 were added 2-tert-butyl-7-dimethylamino-4-methylchromenylium-tetrafluoroborate (Example 8, 65 mg) and potassium acetate (400 mg). The resulting mixture was stirred overnight at ambient temperature before being precipitated into excess ethyl acetate. The crude solid dye was collected by filtration, redissolved in water/acetonitrile and purified by preparative RP-HPLC to give the title dye. MS (MALDI-TOF), M⁺=737. UV/VIS(MeOH): 829 nm.

### Example 24

### 4-(2-{(1E,3E,5E)-5-[2-tert-butyl-7-(dimethylamino)-4H-chromen-4-ylidene]penta-1,3-dienyl}-1-{6-[(2,5-dioxopyrrolidin-1-yl)oxy]-6-oxohexyl}-4,5,6,7-tetrafluoro-3-methyl-3H-indolium-3-yl)butane-1-sulfonate

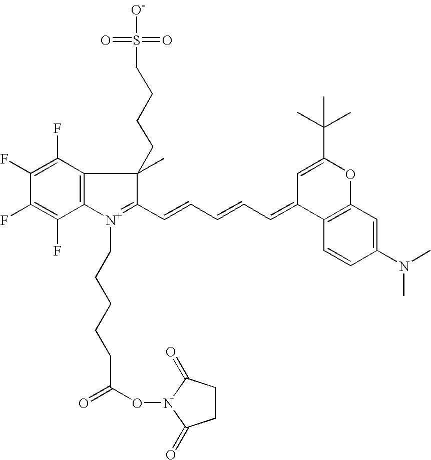

Compound 1 (Example 17, 20 mg) was dissolved in DMF (1 ml) and DMSO (1 ml) with sonication. To the resulting solution were added O—(N-succinimidyl)-N,N,N′,N′-tetramethyluronium tetrafluoroborate (10 mg) and N,N-diisopropylethylamine (5 μl); the reaction was then stirred at ambient temperature for 1 hr. The reaction was confirmed complete by TLC (silica. Methanol, 20: dichloromethane, 80. Rf SM free acid≈0.3, Rf SM NHS ester≈0.5). The mixture was then dripped through a filter into a cooled mixture of ethyl acetate (20 ml) and diethyl ether (20 ml) to give a precipitate. This was collected by centrifugation, washed with fresh ethyl acetate and dried under vacuum to give 25 mg of product. MS (MALDI-TOF), M⁺=896.

### Example 25

### 4-(2-[(1E,3E,5E)-5-(11-tert-butyl-2,3,6,7-tetrahydro-1H,5H,9H-pyrano[2,3-f]pyrido[3,2,1-ij]quinolin-9-ylidene)penta-1,3-dienyl]-1-{6-[(2,5-dioxopyrrolidin-1-yl)oxy]-6-oxohexyl}-4,5,6,7-tetrafluoro-3-methyl-3H-indolium-3-yl)butane-1-sulfonate

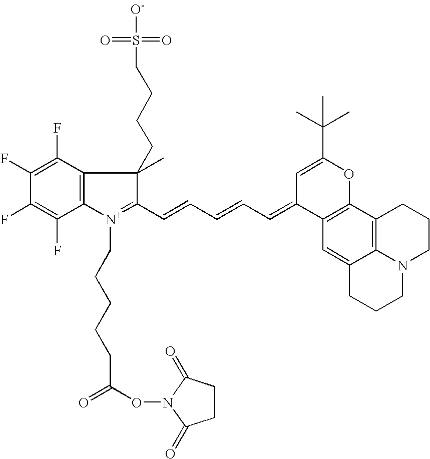

The carboxy dye 4-[2-[(1E,3E,5E)-5-(11-tert-butyl-2,3,6,7-tetrahydro-1H,5H,9H-pyrano[2,3-f]pyrido[3,2,1-ij]quinolin-9-ylidene)penta-1,3-dienyl]-1-(5-carboxypentyl)-4,5,6,7-tetrafluoro-3-methyl-3H-indolium-3-yl]butane-1-sulfonate (Example 18, 16 mg) was dissolved in DMF (1 ml) with sonication. To the resulting solution were added O—(N-succinimidyl)-N,N,N′,N′-tetramethyluronium tetrafluoroborate (10 mg) and N,N-diisopropylethylamine (5 μl); the reaction was then stirred at ambient temperature for 1 hr. The reaction was confirmed complete by TLC (silica. Methanol, 20: dichloromethane, 80. Rf SM free acid≈0.25, Rf SM NHS ester≈0.5). The mixture was then dripped through a filter into a cooled mixture of ethyl acetate (20 ml) and diethyl ether (20 ml) to give a precipitate. This was collected by centrifugation, washed with fresh ethyl acetate and dried under vacuum to give 18 mg of product. MS (MALDI-TOF), M⁺=844.

### Example 26

### Photostability of Dyes of the Invention

Photostability studies were performed as detailed below. Each test dye was dissolved in either a 1:1 mixture of methanol:water or water to give an absorbance reading between 0.7 and 1.2Au. Each solution was then divided into 2 further vials. One vial of dye solution was maintained in a dark environment as a control sample (“dark controls”) during the course of the experiment. The other was exposed to a strong light source (a Wallac light box; 1295-013). Samples were maintained at 22 cm above the light source, with continuous exposure to light. The UV/Visible spectrum of each sample was measured at regular intervals over a six day trial period. The same cuvettes and spectrophotometer were used for each measurement point. For the control samples maintained in the dark, the UV/visible absorption spectra were measured at both the start and at the end of the experiment. Each test was performed in duplicate.

The photostability of Compound 1 (the Cy5F pyrylium dye of Example 17) was studied in comparison with the corresponding non-fluorinated analogue, Cy5 pyrylium (Compound A):

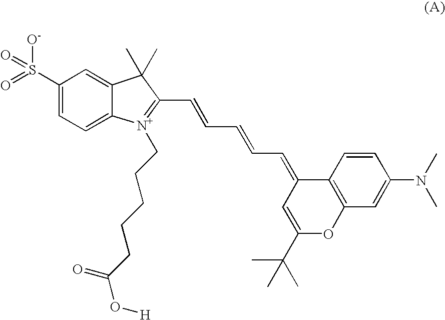

The photostability of Compound 2 (the Cy5F quinolinium dye of Example 12) was studied in comparison with the non-fluorinated analogue, Cy5 quinolinium (Compound B)

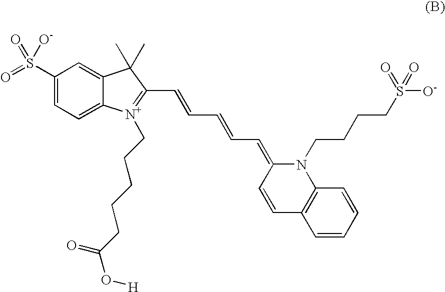

The data for each experiment were normalised and plotted as shown in FIGS. 1 and 2.

In a further example the photostability of the fluorescence signal was measured. The photostability of Compound 3 (the fluorinated Cy3F pyrylium of Example 19) was studied in comparison with a non-fluorinated analogue, Cy3 pyrylium (Compound C)

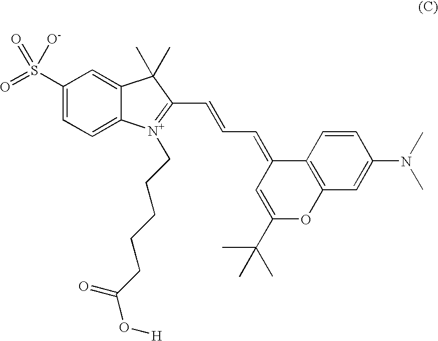

The dyes were dissolved in a 1:1 mixture of methanol:water to give an absorbance reading of 1Au at the dyes lambda max and then diluted 20 times for fluorescence measurements. The samples were exposed to light with a Karl Storz Xenon 175 light source at 100% intensity for 30 min. The cuvette was kept in a water bath while exposed to light, to avoid heating. Fluorescence measurements were made on Cary Eclipse (Varian) fluorescence spectrophotometer 1 cm cuvette, ex/em slit 5 nm

Compound 3 showed improved photostability 0.94 of the dark control while non fluorinated analogue (Compound C) had a max fluorescence intensity 0.79 of the control.

### Example 27

### Synthesis of Dye-RGD Peptide Conjugates (Conjugate 1 and Conjugate 2)

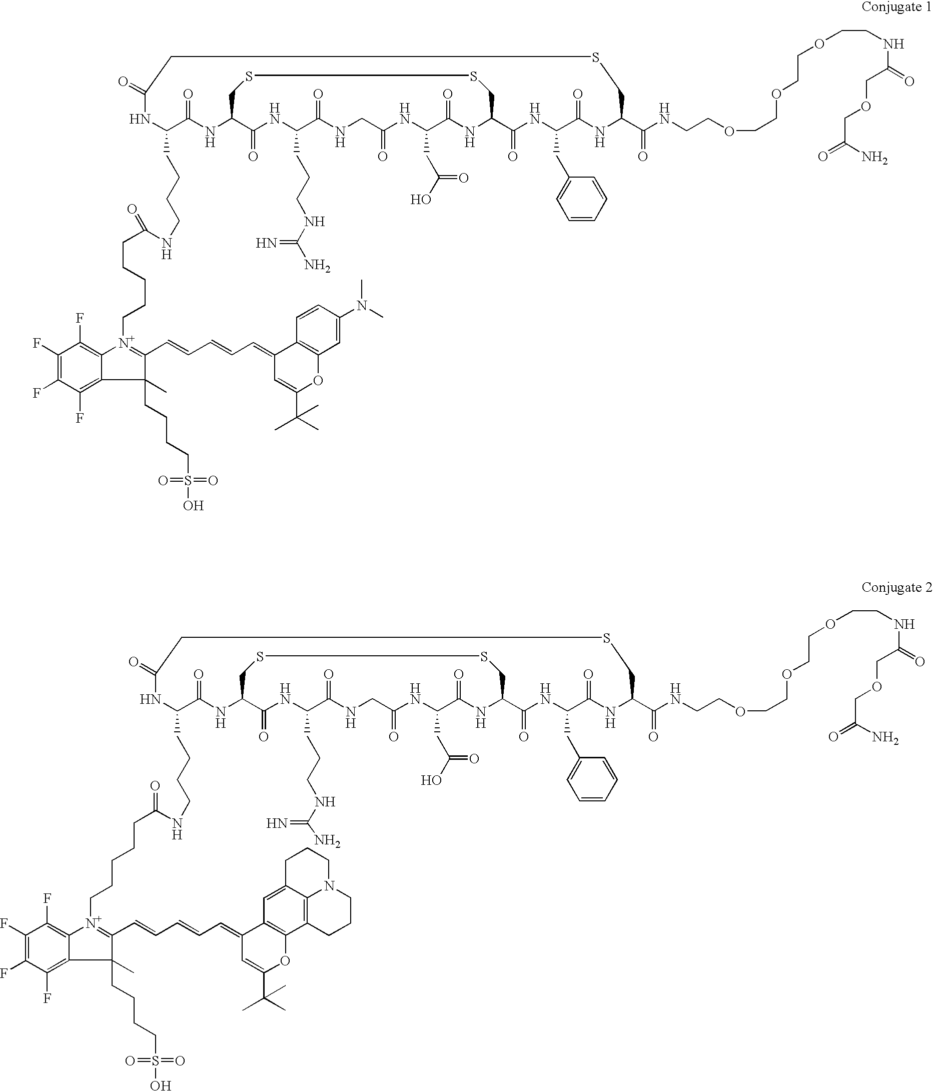

The peptide shown was assembled using standard solid phase peptide synthesis methods. The chloroacetylated peptide was cleaved from the solid support and cyclised in solution, first forming the thioether bridge and then the disulphide bridge.

Compound 1 (Example 17; 0.0018 mmol) was dissolved in NMP (0.5 mL) and NMM (1 μL) was added followed by HATU (0.84 mg, 0.0022 mmol). The solution was stirred in the dark for 5 minutes and then added to a solution of RGD peptide amine (2.3 mg, 0.0018 mmol) in NMP (0.5 mL). The reaction mixture was stirred for five hours at room temperature then heated to 50° C. for two hours.

Conjugate 2 was prepared in a similar manner using the dye of Example 18.

The dye peptide conjugates were detected by LCMS (ESI+, Phenomenex Gemini 50×4.6 mm, 5 micron, 110 Å, A=water/0.1% formic acid, B=MeCN/0.1% formic acid, gradient 5-95 over 10 minutes). Conjugate 1 had retention time=5.73, m/z=994 [MH]²⁺, Conjugate 2 had retention time=6.10, m/z=1020 [MH]²⁺. For conjugate 1, the reaction mixture was then diluted with 5% MeCN/water (5 mL) and the product purified using preparative HPLC.

### Purification and Characterisation

Purification by preparative HPLC (gradient: 5-60% B over 40 min where A=H₂O/0.1% HCOOH and B=MeCN/0.1% HCOOH, flow rate: 10 mL/min, column: Phenomenex Luna 5μ C18 (2) 150×21.20 mm, detection: UV 650 nm, product retention time: 32.60 min) pure dye-peptide conjugate.

The pure product was analysed by analytical LCMS (ESI+, Phenomenex Gemini 50×4.6 mm, 5 micron, 110 Å, A=water/0.1% formic acid, B=MeCN/0.1% formic acid, gradient 5-95 over 10 minutes). Conjugate 1 had retention time=5.73, m/z=994 [MH]²⁺.

1.-35. (canceled)

36. A dye conjugate of Formula I:
[BTM]-(L)ⱼ-Cy^{D} (I)
where:
BTM is a biological targeting moiety;
Cy^{D} is a cyanine dye of Formula II: 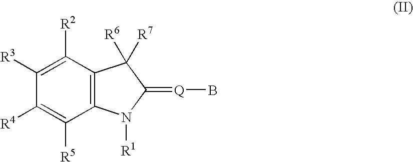
wherein:
Q is a group containing 1, 2 or 3 carbon-carbon double bonds which forms a conjugated system with B;
R¹, R⁶ and R⁷ are selected independently from C₁₋₄ alkyl or —(CH₂)ₖ—SO₃M¹;
R², R³, R⁴ and R⁵ are selected independently from H, F, —SO₃M¹ and —(CF₂)ₘ—F, where m is an integer of value 1 to 4;
M¹ is H or B^{c}, where B^{c} is a biocompatible cation;
j is 0 or 1;
k is an integer of value 1 to 10;
L is a synthetic linker group which is chosen from:
(i) a peptide chain of 1 to 10 amino acid residues;
(ii) a monodisperse polyethyleneglycol structures of Formulae Bio1 or Bio2 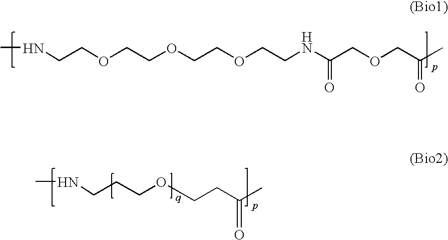
where p is p is an integer from 1 to 10 and q is an integer from 3 to 15;
(iii) a group of formula —(CHR′)ₚ-M-(CHR′)ᵣ—
where M is selected from: —CHR′—, —NR′—, —O—, —S—, —Ar—, —C(O)—NR′— and —C(O)—O—; R′ is H or C₁₋₄ alkyl, Ar is phenylene, optionally substituted with sulphonate, p and r are integers of value 1-5;
B is an aromatic chromophore selected from benzo[b]pyrilium, quinolinium and acridinium chromophores;
with the proviso that at least one of R², R³, R⁴ and R⁵ is F or —(CF₂)ₘ—F.

37. The conjugate of claim 36, wherein Q is the group: 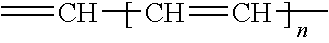
wherein n=1, 2 or 3.

38. The conjugate of claim 36, where B is of Formula IIa: 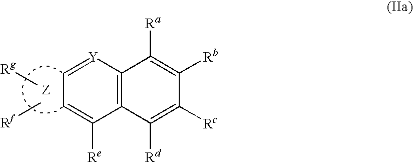
wherein:
Y is selected from O⁺ and N⁺—R⁸, where R⁸ is selected from H, C₁₋₄ alkyl and —(CH₂)ₖ—SO₃M¹;
R^{a}, R^{b}, R^{c}, R^{d}, R^{e}, R^{f} and R^{g} are selected independently from Q, H, C₁₋₄ alkyl, C₆₋₁₀ aryl, heteroaryl, aralkyl, Hal, sulphydryl, amino, C₁₋₄ alkyl-substituted amino, quaternary ammonium, —SO₃M¹, —OR⁹ and —COOR⁹, where R⁹ is selected from H and C₁₋₄ alkyl;
Z represents an optional fused phenyl ring, such that R^{f} and R^{g} are attached to the Z ring when Z is present, or the Y ring when Z is absent;
with the proviso that one of R^{a}, R^{b}, R^{c}, R^{d}, R^{e}, R^{f} and R^{g} is Q.

39. The conjugate of claim 38, where Y is O⁺, Z is absent, and R^{e} is 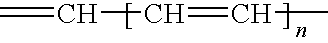
wherein n=1, 2 or 3, said dye having the Formula (III):

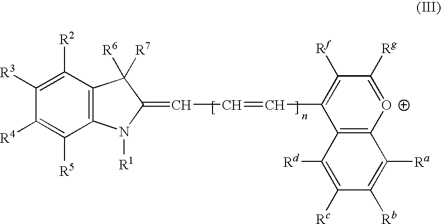

40. The conjugate of claim 38, where Y is N⁺—R⁸, Z is absent, and the Q group is as defined is 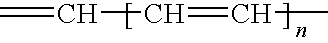
wherein n=1, 2 or 3,
said dye having a structure chosen from Formula (IVa) or (IVb) or (IVc):

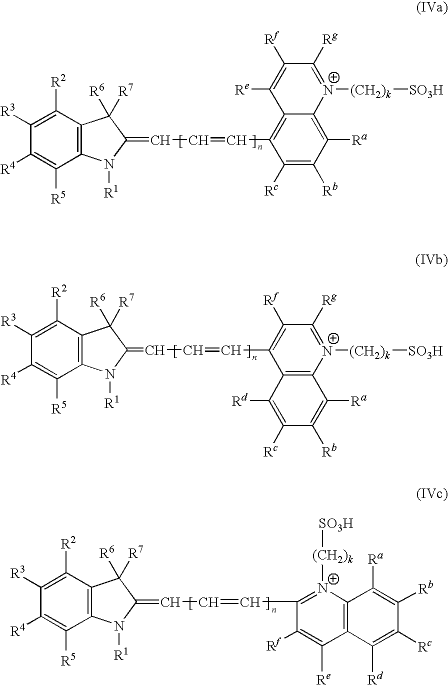

41. The conjugate of claim 38, where Y is N⁺—R⁵, Z is present, and the Q group is as defined in 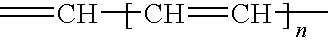
wherein n=1, 2 or 3, said dye having the Formula (V):

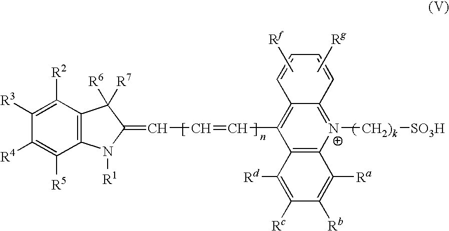

42. The conjugate of claim 36, wherein at least one of R², R³, R⁴ and R⁵ is F.

43. The conjugate of claim 36, where BTM is chosen from:
(i) a 3-100 mer peptide;
(ii) an enzyme substrate, enzyme antagonists or enzyme inhibitor;
(iii) a receptor-binding compound;
(iv) an oligonucleotide;
(v) an oligo-DNA or oligo-RNA fragment.

44. A pharmaceutical composition which comprises the conjugate of claim 36 together with a biocompatible carrier, in a form suitable for mammalian administration.

45. A kit for the preparation of the pharmaceutical composition which comprises the conjugate of claim 36 together with a biocompatible carrier, in a form suitable for mammalian administration., said kit comprising the conjugate of claim 36 in sterile, solid form such that upon reconstitution with a sterile supply of a biocompatible carrier, dissolution occurs to give the desired pharmaceutical composition.

46. A functionalised dye useful in the preparation of the conjugate of claim 36, which comprises the Cy^{D} of Formula II as defined in claim 36 wherein said Cy^{D} further comprises a group Q^{a}, where Q^{a} is a reactive functional group suitable for conjugation to the BTM as defined in claim 36, and Q^{a} is selected from: carboxyl, an activated ester; isothiocyanate; maleimide; haloacetamide; hydrazide; dichlorotriazine or phosphoramidite.

47. A method of preparation of the conjugate of claim 36, which comprises:
(i) mixing the BTM as defined in claim 36 with the Q^{a}-functionalised Cy^{D} of Formula II as defined in claim 36 wherein said Cy^{D} further comprises a group Q^{a}, where Q^{a} is a reactive functional group suitable for conjugation to the BTM as defined in claim 36, and Q^{a} is selected from: carboxyl, an activated ester;
isothiocyanate; maleimide; haloacetamide; hydrazide; dichlorotriazine or phosphoramidite;
(ii) incubating said Q^{a}-functionalised Cy^{D} with said BTM under conditions suitable for reaction of the Q^{a} group with the BTM, to give the desired conjugate;
(iii) optional separation and/or purification of the conjugate from the reaction mixture of step (ii).

48. A method of in vivo optical imaging of the mammalian body which comprises use of either the conjugate of claim 36 or the pharmaceutical composition which comprises the conjugate of claim 36 together with a biocompatible carrier, in a form suitable for mammalian administration to obtain images of sites of localisation of the BTM in vivo.

49. The method of claim 48, which comprises the steps of:
(i) a tissue surface of interest within the mammalian body is illuminated with an excitation light;
(ii) fluorescence from the imaging agent, which is generated by excitation of the Cy^{D} is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

50. The method of claim 49 which comprises:
(a) exposing light-scattering biologic tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

51. A method of detection, staging, diagnosis, monitoring of disease progression or monitoring of treatment of a disease state of the mammalian body which comprises the in vivo optical imaging method of claim 48.

52. A method for the assay of an analyte in a sample which method comprises:
(i) contacting the analyte with a specific binding partner for said analyte under conditions suitable to cause the binding of at least a portion of said analyte to said specific binding partner to form a complex, wherein said specific binding partner comprises the dye conjugate of claim 36;
(ii) measuring the emitted fluorescence of the labelled complex; and
(iii) correlating the emitted fluorescence with the presence or the amount of said analyte in said sample.
